(19) United States
(12) Patent Application Publication
(10) Pub. No.: US 2007/0003583 A1
(43) Pub. Date: Jan. 4, 2007

(54) Cosmetic compositions comprising cyclohexanepolycarboxylic acid derivatives

(75) Inventors: Uwe Storzum, Worms (DE); Boris Breitscheidel, Fulda (DE)

Correspondence Address: CONNOLLY BOVE LODGE & HUTZ, LLP, P O BOX 2207, WILMINGTON, DE, 19899, US

(73) Assignee: Basf Aktiengesellschaft, Ludwigshafen (DE)

(21) Appl. No.: 10/555,573

(22) PCT Filed: May 10, 2004
(86) PCT No.: PCT/EP04/04952

§ 371 Date: Nov. 4, 2005

(30) Foreign Application Priority Data
May 9, 2003 (EP) 03010468.1

(51) Int. Cl. A61K 8/37

(52) U.S. Cl. 424/401

(57) ABSTRACT

The present invention relates to cosmetic compositions, in particular fragrance compositions and cosmetic compositions for decorative application on the human body, comprising at least one cyclohexanepolycarboxylic acid derivative, and to the use of cyclohexanepolycarboxylic acid derivatives in cosmetic compositions.

The present invention relates to cosmetic compositions, in particular fragrance compositions and cosmetic compositions for decorative application on the human body, comprising at least one cyclohexanepolycarboxylic acid derivative, and to the use of cyclohexanepolycarboxylic acid derivatives in cosmetic compositions.

Cosmetic compositions of all types in many cases comprise polycarboxylic acid derivatives, in particular aromatic polycarboxylic acid derivatives for various application purposes.

For example, polycarboxylic esters are used in fragrance compositions.

EP-A-0 684 037 relates to deodorizing compositions which comprise a fragrance composition and an inorganic deodorizing material. The fragrance composition preferably comprises esters, in particular diethyl phthalate.

WO 02/13776 relates to a deodorant comprising an active deodorant, gelling agent based on soap, a fragrance composition, an agent that cools on the skin which is solubilized in a solvent system comprising a nonaqueous liquid carrier and water. The fragrance composition comprises esters such as diethyl phthalate.

EP-A-1 068 862 discloses a deodorizing active ingredient combination comprising a polyhydric alcohol, in particular a 1,2-diol, and at least one trialkyl ester of citric acid.

Furthermore, the use of polycarboxylic esters in cosmetic compositions for decorative application on the human body, for example in nail varnishes and cosmetic sticks, is known.

For example, DE 200 09 445 U1 relates to cosmetic compositions, preferably in the form of sticks, such as lipsticks, lipcare sticks, concealing sticks, etc., where the oil component used is, for example, esters of alkanedicarboxylic acids.

US 2001/000 7676 A1 relates to nail varnish compositions which comprise, for example, dibutyl phthalate as plasticizer.

Since phthalates can have an allergy-triggering effect, U.S. Pat. No. 5,882,636 discloses nail varnish compositions which are phthalate-free and instead comprise, for example, dibutyl phthalate adipate, pentaerythiryl tetrabenzoate, 2,2,4-trimethyl-1,3-pentanediol diisobutyrate, pentaerythiryl tetraacetate and mixtures thereof.

It is an object of the present invention to provide cosmetic compositions which comprise polycarboxylic acid derivatives which are nontoxic and do not trigger allergies, it being possible, for example, for these polycarboxylic acid derivatives to be used as a replacement for the phthalates which possibly trigger allergies.

This object is achieved through cosmetic compositions comprising at least one cyclohexanepolycarboxylic acid derivative.

The term “cosmetic composition” is here to be understood in a very wide sense. For the purposes of the present application, the term comprises both fragrance compositions and also cosmetic compositions of other types, such as cleansing and care compositions for skin and hair and compositions for decorative application on the human body. It is also possible for a fragrance composition comprising at least one cyclohexanepolycarboxylic acid derivative to be used for the preparation of perfumed products chosen from perfume, Eau de Toilette and the like, soap, shower gel, bath gel, shampoo, further haircare compositions, cosmetic preparations such as creams and lotions or sunscreen compositions and also body and room deodorants or detergents or care compositions for textiles.

The cyclohexanepolycarboxylic acid derivatives can here be effective in the cosmetic compositions in diverse ways. For example, they can, for example in fragrance compositions, serve as solvents, for example as solvents for fragrances such as musk fragrances. Furthermore, the cyclohexanepolycarboxylic acid derivative can, for example, serve as plasticizers in nail varnish compositions.

Preferably, the cosmetic compositions according to the invention have at least one cyclohexanepolycarboxylic acid derivative of the formula (I) 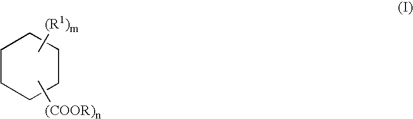
in which
R¹ is C₁-C₁₀-alkyl or C₃-C₈-cycloalkyl,
m is 0, 1, 2 or 3,
n is 2, 3 or 4, and
R is hydrogen or C₁-C₃₀-alkyl, where at least one radical R is C₁-C₃₀-alkyl.

If m is 2 or 3, the radicals R¹ may be identical or different. The C₁-C₁₀-alkyl groups and the C₁-C₃₀-alkyl groups may be straight-chain or branched. If R¹ is an alkyl group, it is preferably a C₁-C₈-alkyl group, particularly preferably a C₁-C₆-alkyl group. Examples of such alkyl groups are methyl, ethyl, n-propyl, isopropyl, n-butyl, isobutyl, sec-butyl, tert-butyl, n-pentyl, n-hexyl, n-octyl and 2-ethylhexyl.

The m radicals R may be identical or different. R is preferably C₂-C₂₀-alkyl, particularly preferably C₃-C₁₈-alkyl. Examples of such alkyl groups are the alkyl groups already specified for R¹, and n-nonyl, isononyl, n-decyl, isodecyl, n-undecyl, isoundecyl, n-dodecyl, isododecyl, n-tridecyl, isotridecyl, stearyl, and n-eicosyl.

The cyclohexanepolycarboxylic acid derivatives comprised according to the invention are, in particular, mono-, di-, tri-, tetraesters and anhydrides of cyclohexanepolycarboxylic acids. Preferably, all of the carboxylic acid groups are esterified. The esters used are alkyl, cycloalkyl and alkoxyalkyl esters, where the alkyl, cycloalkyl and alkoxyalkyl groups generally comprise 1 to 30, preferably 2 to 20 and particularly preferably 3 to 18, carbon atoms and may be branched or linear.

Preferably, the at least one cyclohexanepolycarboxylic acid derivative is chosen from the group consisting of ring-hydrogenated mono- and dialkyl esters of phthalic acid, isophthalic acid and terephthalic acid, ring-hydrogenated mono-, di- and trialkyl esters of trimellitic acid, of trimesic acid and of hemimellitic acid, or mono-, di-, tri- and tetraalkyl esters of pyrromellitic acid, where the alkyl groups may be linear or branched and in each case have 1 to 30, preferably 2 to 10, particularly preferably 3 to 18, carbon atoms, and mixtures of two or more thereof.

Particular preference is given to alkyl esters of cyclohexane-1,4-dicarboxylic acid, such as, for example, monomethyl ester of cyclohexane-1,4-dicarboxylic acid, dimethyl ester of cyclohexane-1,4-dicarboxylic acid, diethyl ester of cyclohexane-1,4-dicarboxylic acid, di-n-propyl ester of cyclohexane-1,4-dicarboxylic acid, di-n-butyl ester of cyclohexane-1,4-dicarboxylic acid, di-tert-butyl ester of cyclohexane-1,4-dicarboxylic acid, diisobutyl ester of cyclohexane-1,4-dicarboxylic acid, monoglycol ester of cyclohexane-1,4-dicarboxylic acid, diglycol ester of cyclohexane-1,4-dicarboxylic acid, di-n-octyl ester of cyclohexane-1,4-dicarboxylic acid, diisooctyl ester of cyclohexane-1,4-dicarboxylic acid, mono-2-ethylhexyl ester of cyclohexane-1,4-dicarboxylic acid, di-2-ethylhexyl ester of cyclohexane-1,4-dicarboxylic acid, di-n-nonyl ester of cyclohexane-1,4-dicarboxylic acid, diisononyl ester of cyclohexane-1,4-dicarboxylic acid, di-n-decyl ester of cyclohexane-1,4-dicarboxylic acid, di-n-undecyl ester of cyclohexane-1,4-dicarboxylic acid, diisodecyl ester of cyclohexane-1,4-dicarboxylic acid, diisododecyl ester of cyclohexane-1,4-dicarboxylic acid, di-n-octadecyl ester of cyclohexane-1,4-dicarboxylic acid, diisooctadecyl ester of cyclohexane-1,4-dicarboxylic acid, di-n-eicosyl ester of cyclohexane-1,4-dicarboxylic acid, monocyclohexyl ester of cyclohexane-1,4-dicarboxylic acid, dicyclohexyl ester of cyclohexane-1,4-dicarboxylic acid;

diisopropyl ester of cyclohexane-1,4-dicarboxylic acid, di-n-hexyl ester of cyclohexane-1,4-dicarboxylic acid, diisohexyl ester of cyclohexane-1,4-dicarboxylic acid, di-n-heptyl ester of cyclohexane-1,4-dicarboxylic acid, diisoheptyl ester of cyclohexane-1,4-dicarboxylic acid, di-2-propylheptyl ester of cyclohexane-1,4-dicarboxylic acid, diisoundecyl ester of cyclohexane-1,4-dicarboxylic acid, di-n-dodecyl ester of cyclohexane-1,4-dicarboxylic acid, di-n-tridecyl ester of cyclohexane-1,4-dicarboxylic acid, diisotridecyl ester of cyclohexane-1,4-dicarboxylic acid, di-n-pentyl ester of cyclohexane-1,4-dicarboxylic acid, diisopentyl ester of cyclohexane-1,4-dicarboxylic acid;

diisopropyl ester of cyclohexane-1,2-dicarboxylic acid, di-n-hexyl ester of cyclohexane-1,2-dicarboxylic acid, diisohexyl ester of cyclohexane-1,2-dicarboxylic acid, di-n-heptyl ester of cyclohexane-1,2-dicarboxylic acid, diisoheptyl ester of cyclohexane-1,2-dicarboxylic acid, di-2-propylheptyl ester of cyclohexane-1,2-dicarboxylic acid, diisoundecyl ester of cyclohexane-1,2-dicarboxylic acid, di-n-dodecyl ester of cyclohexane-1,2-dicarboxylic acid, di-n-tridecyl ester of cyclohexane-1,2-dicarboxylic acid, diisotridecyl ester of cyclohexane-1,2-dicarboxylic acid, di-n-pentyl ester of cyclohexane-1,2-dicarboxylic acid, diisopentyl ester of cyclohexane-1,2-dicarboxylic acid;

alkyl esters of cyclohexane-1,2-dicarboxylic acid, such as, for example, monomethyl ester of cyclohexane-1,2-dicarboxylic acid, dimethyl ester of cyclohexane-1,2-dicarboxylic acid, diethyl ester of cyclohexane-1,2-dicarboxylic acid, di-n-propyl ester of cyclohexane-1,2-dicarboxylic acid, di-n-butyl ester of cyclohexane-1,2-dicarboxylic acid, di-tert-butyl ester of cyclohexane-1,2-dicarboxylic acid, diisobutyl ester of cyclohexane-1,2-dicarboxylic acid, monoglycol ester of cyclohexane-1,2-dicarboxylic acid, diglycol ester of cyclohexane-1,2-dicarboxylic acid, di-n-octyl ester of cyclohexane-1,2-dicarboxylic acid, diisooctyl ester of cyclohexane-1,2-dicarboxylic acid, di-2-ethylhexyl ester of cyclohexane-1,2-dicarboxylic acid, di-n-nonyl ester of cyclohexane-1,2-dicarboxylic acid, diisononyl ester of cyclohexane-1,2-dicarboxylic acid, di-n-decyl ester of cyclohexane-1,2-dicarboxylic acid, diisodecyl ester of cyclohexane-1,2-dicarboxylic acid, di-n-undecyl ester of cyclohexane-1,2-dicarboxylic acid, diisododecyl ester of cyclohexane-1,2-dicarboxylic acid, di-n-octadecyl ester of cyclohexane-1,2-dicarboxylic acid, diisooctadecyl ester of cyclohexane-1,2-dicarboxylic acid, di-n-eicosyl ester of cyclohexane-1,2-dicarboxylic acid, monocyclohexyl ester of cyclohexane-1,2-dicarboxylic acid, dicyclohexyl ester of cyclohexane-1,2-dicarboxylic acid;

mixed esters of cyclohexane-1,2-dicarboxylic acid with C₁ to C₁₃ alcohols, such as, for example, ethyl methyl ester of cyclohexane-1,2-dicarboxylic acid, n-propyl methyl ester of cyclohexane-1,2-dicarboxylic acid, isopropyl methyl ester of cyclohexane-1,2-dicarboxylic acid, n-butyl methyl ester of cyclohexane-1,2-dicarboxylic acid, tert-butyl methyl ester of cyclohexane-1,2-dicarboxylic acid, isobutyl methyl ester of cyclohexane-1,2-dicarboxylic acid, glycol methyl ester of cyclohexane-1,2-dicarboxylic acid, n-hexyl methyl ester of cyclohexane-1,2-dicarboxylic acid, isohexyl methyl ester of cyclohexane-1,2-dicarboxylic acid, n-heptyl methyl ester of cyclohexane-1,2-dicarboxylic acid, isoheptyl methyl ester of cyclohexane-1,2-dicarboxylic acid, n-octyl methyl ester of cyclohexane-1,2-dicarboxylic acid, isooctyl methyl ester of cyclohexane-1,2-dicarboxylic acid, 2-ethylhexyl methyl ester of cyclohexane-1,2-dicarboxylic acid, n-nonyl methyl ester of cyclohexane-1,2-dicarboxylic acid, isononyl methyl ester of cyclohexane-1,2-dicarboxylic acid, 2-propylheptyl methyl ester of cyclohexane-1,2-dicarboxylic acid, n-decyl methyl ester of cyclohexane-1,2-dicarboxylic acid, isodecyl methyl ester of cyclohexane-1,2-dicarboxylic acid, n-undecyl methyl ester of cyclohexane-1,2-dicarboxylic acid, isoundecyl methyl ester of cyclohexane-1,2-dicarboxylic acid, n-dodecyl methyl ester of cyclohexane-1,2-dicarboxylic acid, isododecyl methyl ester of cyclohexane-1,2-dicarboxylic acid, n-tridecyl methyl ester of cyclohexane-1,2-dicarboxylic acid, isotridecyl methyl ester of cyclohexane-1,2-dicarboxylic acid, n-propyl ethyl ester of cyclohexane-1,2-dicarboxylic acid, isopropyl ethyl ester of cyclohexane-1,2-dicarboxylic acid, n-butyl ethyl ester of cyclohexane-1,2-dicarboxylic acid, tert-butyl ethyl ester of cyclohexane-1,2-dicarboxylic acid, isobutyl ethyl ester of cyclohexane-1,2-dicarboxylic acid, glycol ethyl ester of cyclohexane-1,2-dicarboxylic acid, n-hexyl ethyl ester of cyclohexane-1,2-dicarboxylic acid, isohexyl ethyl ester of cyclohexane-1,2-dicarboxylic acid, n-heptyl ethyl ester of cyclohexane-1,2-dicarboxylic acid, isoheptyl ethyl ester of cyclohexane-1,2-dicarboxylic acid, n-octyl ethyl ester of cyclohexane-1,2-dicarboxylic acid, isooctyl ethyl ester of cyclohexane-1,2-dicarboxylic acid, 2-ethylhexyl ethyl ester of cyclohexane-1,2-dicarboxylic acid, n-nonyl ethyl ester of cyclohexane-1,2-dicarboxylic acid, isononyl ethyl ester of cyclohexane-1,2-dicarboxylic acid, 2-propylheptyl ethyl ester of cyclohexane-1,2-dicarboxylic acid, n-decyl ethyl ester of cyclohexane-1,2-dicarboxylic acid, isodecyl ethyl ester of cyclohexane-1,2-dicarboxylic acid, n-undecyl ethyl ester of cyclohexane-1,2-dicarboxylic acid, isoundecyl ethyl ester of cyclohexane-1,2-dicarboxylic acid, n-dodecyl ethyl ester of cyclohexane-1,2-dicarboxylic acid, isododecyl ethyl ester of cyclohexane-1,2-dicarboxylic acid, n-tridecyl ethyl ester of cyclohexane-1,2-dicarboxylic acid, isotridecyl ethyl ester of cyclohexane-1,2-dicarboxylic acid, isopropyl n-propyl ester of cyclohexane-1,2-dicarboxylic acid, n-butyl n-propyl ester of cyclohexane-1,2-dicarboxylic acid, tert-butyl n-propyl ester of cyclohexane-1,2-dicarboxylic acid, isobutyl n-propyl ester of cyclohexane-1,2-dicarboxylic acid, glycol n-propyl ester of cyclohexane-1,2-dicarboxylic acid, n-hexyl n-propyl ester of cyclohexane-1,2-dicarboxylic acid, isohexyl n-propyl ester of cyclohexane-1,2-dicarboxylic acid, n-heptyl n-propyl ester of cyclohexane-1,2-dicarboxylic acid, isoheptyl n-propyl ester of cyclohexane-1,2-dicarboxylic acid, n-octyl n-propyl ester of cyclohexane-1,2-dicarboxylic acid, isooctyl n-propyl ester of cyclohexane-1,2-dicarboxylic acid, 2-ethylhexyl n-propyl ester of cyclohexane-1,2-dicarboxylic acid, n-nonyl n-propyl ester of cyclohexane-1,2-dicarboxylic acid, isononyl n-propyl ester of cyclohexane-1,2-dicarboxylic acid, 2-propylheptyl n-propyl ester of cyclohexane-1,2-dicarboxylic acid, n-decyl n-propyl ester of cyclohexane-1,2-dicarboxylic acid, isodecyl n-propyl ester of cyclohexane-1,2-dicarboxylic acid, n-undecyl n-propyl ester of cyclohexane-1,2-dicarboxylic acid, isoundecyl n-propyl ester of cyclohexane-1,2-dicarboxylic acid, n-dodecyl n-propyl ester of cyclohexane-1,2-dicarboxylic acid, isododecyl n-propyl ester of cyclohexane-1,2-dicarboxylic acid, n-tridecyl n-propyl ester of cyclohexane-1,2-dicarboxylic acid, isotridecyl n-propyl ester of cyclohexane-1,2-dicarboxylic acid, n-butyl isopropyl ester of cyclohexane-1,2-dicarboxylic acid, tert-butyl isopropyl ester of cyclohexane-1,2-dicarboxylic acid, isobutyl isopropyl ester of cyclohexane-1,2-dicarboxylic acid, glycol isopropyl ester of cyclohexane-1,2-dicarboxylic acid, n-hexyl isopropyl ester of cyclohexane-1,2-dicarboxylic acid, isohexyl isopropyl ester of cyclohexane-1,2-dicarboxylic acid, n-heptyl isopropyl ester of cyclohexane-1,2-dicarboxylic acid, isoheptyl isopropyl ester of cyclohexane-1,2-dicarboxylic acid, n-octyl isopropyl ester of cyclohexane-1,2-dicarboxylic acid, isooctyl isopropyl ester of cyclohexane-1,2-dicarboxylic acid, 2-ethylhexyl isopropyl ester of cyclohexane-1,2-dicarboxylic acid, n-nonyl isopropyl ester of cyclohexane-1,2-dicarboxylic acid, isononyl isopropyl ester of cyclohexane-1,2-dicarboxylic acid, 2-propylheptyl isopropyl ester of cyclohexane-1,2-dicarboxylic acid, n-decyl isopropyl ester of cyclohexane-1,2-dicarboxylic acid, isodecyl isopropyl ester of cyclohexane-1,2-dicarboxylic acid, n-undecyl isopropyl ester of cyclohexane-1,2-dicarboxylic acid, isoundecyl isopropyl ester of cyclohexane-1,2-dicarboxylic acid, n-dodecyl isopropyl ester of cyclohexane-1,2-dicarboxylic acid, isododecyl isopropyl ester of cyclohexane-1,2-dicarboxylic acid, n-tridecyl isopropyl ester of cyclohexane-1,2-dicarboxylic acid, isotridecyl isopropyl ester of cyclohexane-1,2-dicarboxylic acid, tert-butyl n-butyl ester of cyclohexane-1,2-dicarboxylic acid, isobutyl n-butyl ester of cyclohexane-1,2-dicarboxylic acid, glycol n-butyl ester of cyclohexane-1,2-dicarboxylic acid, n-hexyl n-butyl ester of cyclohexane-1,2-dicarboxylic acid, isohexyl n-butyl ester of cyclohexane-1,2-dicarboxylic acid, n-heptyl n-butyl ester of cyclohexane-1,2-dicarboxylic acid, isoheptyl n-butyl ester of cyclohexane-1,2-dicarboxylic acid, n-octyl n-butyl ester of cyclohexane-1,2-dicarboxylic acid, isooctyl n-butyl ester of cyclohexane-1,2-dicarboxylic acid, 2-ethylhexyl n-butyl ester of cyclohexane-1,2-dicarboxylic acid, n-nonyl n-butyl ester of cyclohexane-1,2-dicarboxylic acid, isononyl n-butyl ester of cyclohexane-1,2-dicarboxylic acid, 2-propylheptyl n-butyl ester of cyclohexane-1,2-dicarboxylic acid, n-decyl n-butyl ester of cyclohexane-1,2-dicarboxylic acid, isodecyl n-butyl ester of cyclohexane-1,2-dicarboxylic acid, n-undecyl n-butyl ester of cyclohexane-1,2-dicarboxylic acid, isoundecyl n-butyl ester of cyclohexane-1,2-dicarboxylic acid, n-dodecyl n-butyl ester of cyclohexane-1,2-dicarboxylic acid, isododecyl n-butyl ester of cyclohexane-1,2-dicarboxylic acid, n-tridecyl n-butyl ester of cyclohexane-1,2-dicarboxylic acid, isotridecyl n-butyl ester of cyclohexane-1,2-dicarboxylic acid, isobutyl tert-butyl ester of cyclohexane-1,2-dicarboxylic acid, glycol tert-butyl ester of cyclohexane-1,2-dicarboxylic acid, n-hexyl tert-butyl ester of cyclohexane-1,2-dicarboxylic acid, isohexyl tert-butyl ester of cyclohexane-1,2-dicarboxylic acid, n-heptyl tert-butyl ester of cyclohexane-1,2-dicarboxylic acid, isoheptyl tert-butyl ester of cyclohexane-1,2-dicarboxylic acid, n-octyl tert-butyl ester of cyclohexane-1,2-dicarboxylic acid, isooctyl tert-butyl ester of cyclohexane-1,2-dicarboxylic acid, 2-ethylhexyl tert-butyl ester of cyclohexane-1,2-dicarboxylic acid, n-nonyl tert-butyl ester of cyclohexane-1,2-dicarboxylic acid, isononyl tert-butyl ester of cyclohexane-1,2-dicarboxylic acid, 2-propylheptyl tert-butyl ester of cyclohexane-1,2-dicarboxylic acid, n-decyl tert-butyl ester of cyclohexane-1,2-dicarboxylic acid, isodecyl tert-butyl ester of cyclohexane-1,2-dicarboxylic acid, n-undecyl tert-butyl ester of cyclohexane-1,2-dicarboxylic acid, isoundecyl tert-butyl ester of cyclohexane-1,2-dicarboxylic acid, n-dodecyl tert-butyl ester of cyclohexane-1,2-dicarboxylic acid, isododecyl tert-butyl ester of cyclohexane-1,2-dicarboxylic acid, n-tridecyl tert-butyl ester of cyclohexane-1,2-dicarboxylic acid, isotridecyl tert-butyl ester of cyclohexane-1,2-dicarboxylic acid, glycol isobutyl ester of cyclohexane-1,2-dicarboxylic acid, n-hexyl isobutyl ester of cyclohexane-1,2-dicarboxylic acid, isohexyl isobutyl ester of cyclohexane-1,2-dicarboxylic acid, n-heptyl isobutyl ester of cyclohexane-1,2-dicarboxylic acid, isoheptyl isobutyl ester of cyclohexane-1,2-dicarboxylic acid, n-octyl isobutyl ester of cyclohexane-1,2-dicarboxylic acid, isooctyl isobutyl ester of cyclohexane-1,2-dicarboxylic acid, 2-ethylhexyl isobutyl ester of cyclohexane-1,2-dicarboxylic acid, n-nonyl isobutyl ester of cyclohexane-1,2-dicarboxylic acid, isononyl isobutyl ester of cyclohexane-1,2-dicarboxylic acid, 2-propylheptyl isobutyl ester of cyclohexane-1,2-dicarboxylic acid, n-decyl isobutyl ester of cyclohexane-1,2-dicarboxylic acid, isodecyl isobutyl ester of cyclohexane-1,2-dicarboxylic acid, n-undecyl isobutyl ester of cyclohexane-1,2-dicarboxylic acid, isoundecyl isobutyl ester of cyclohexane-1,2-dicarboxylic acid, n-dodecyl isobutyl ester of cyclohexane-1,2-dicarboxylic acid, isododecyl isobutyl ester of cyclohexane-1,2-dicarboxylic acid, n-tridecyl isobutyl ester of cyclohexane-1,2-dicarboxylic acid, isotridecyl isobutyl ester of cyclohexane-1,2-dicarboxylic acid, n-hexyl glycol ester of cyclohexane-1,2-dicarboxylic acid, isohexyl glycol ester of cyclohexane-1,2-dicarboxylic acid, n-heptyl glycol ester of cyclohexane-1,2-dicarboxylic acid, isoheptyl glycol ester of cyclohexane-1,2-dicarboxylic acid, n-octyl glycol ester of cyclohexane-1,2-dicarboxylic acid, isooctyl glycol ester of cyclohexane-1,2-dicarboxylic acid, 2-ethylhexyl glycol ester of cyclohexane-1,2-dicarboxylic acid, n-nonyl glycol ester of cyclohexane-1,2-dicarboxylic acid, isononyl glycol ester of cyclohexane-1,2-dicarboxylic acid, 2-propylheptyl glycol ester of cyclohexane-1,2-dicarboxylic acid, n-decyl glycol ester of cyclohexane-1,2-dicarboxylic acid, isodecyl glycol ester of cyclohexane-1,2-dicarboxylic acid, n-undecyl glycol ester of cyclohexane-1,2-dicarboxylic acid, isoundecyl glycol ester of cyclohexane-1,2-dicarboxylic acid, n-dodecyl glycol ester of cyclohexane-1,2-dicarboxylic acid, isododecyl glycol ester of cyclohexane-1,2-dicarboxylic acid, n-tridecyl glycol ester of cyclohexane-1,2-dicarboxylic acid, isotridecyl glycol ester of cyclohexane-1,2-dicarboxylic acid, isohexyl n-hexyl ester of cyclohexane-1,2-dicarboxylic acid, n-heptyl n-hexyl ester of cyclohexane-1,2-dicarboxylic acid, isoheptyl n-hexyl ester of cyclohexane-1,2-dicarboxylic acid, n-octyl n-hexyl ester of cyclohexane-1,2-dicarboxylic acid, isooctyl n-hexyl ester of cyclohexane-1,2-dicarboxylic acid, 2-ethylhexyl n-hexyl ester of cyclohexane-1,2-dicarboxylic acid, n-nonyl n-hexyl ester of cyclohexane-1,2-dicarboxylic acid, isononyl n-hexyl ester of cyclohexane-1,2-dicarboxylic acid, 2-propylheptyl n-hexyl ester of cyclohexane-1,2-dicarboxylic acid, n-decyl n-hexyl ester of cyclohexane-1,2-dicarboxylic acid, isodecyl n-hexyl ester of cyclohexane-1,2-dicarboxylic acid, n-undecyl n-hexyl ester of cyclohexane-1,2-dicarboxylic acid, isoundecyl n-hexyl ester of cyclohexane-1,2-dicarboxylic acid, n-dodecyl n-hexyl ester of cyclohexane-1,2-dicarboxylic acid, isododecyl n-hexyl ester of cyclohexane-1,2-dicarboxylic acid, n-tridecyl n-hexyl ester of cyclohexane-1,2-dicarboxylic acid, isotridecyl n-hexyl ester of cyclohexane-1,2-dicarboxylic acid, n-heptyl isohexyl ester of cyclohexane-1,2-dicarboxylic acid, isoheptyl isohexyl ester of cyclohexane-1,2-dicarboxylic acid, n-octyl isohexyl ester of cyclohexane-1,2-dicarboxylic acid, isooctyl isohexyl ester of cyclohexane-1,2-dicarboxylic acid, 2-ethylhexyl isohexyl ester of cyclohexane-1,2-dicarboxylic acid, n-nonyl isohexyl ester of cyclohexane-1,2-dicarboxylic acid, isononyl isohexyl ester of cyclohexane-1,2-dicarboxylic acid, 2-propylheptyl isohexyl ester of cyclohexane-1,2-dicarboxylic acid, n-decyl isohexyl ester of cyclohexane-1,2-dicarboxylic acid, isodecyl isohexyl ester of cyclohexane-1,2-dicarboxylic acid, n-undecyl isohexyl ester of cyclohexane-1,2-dicarboxylic acid, isoundecyl isohexyl ester of cyclohexane-1,2-dicarboxylic acid, n-dodecyl isohexyl ester of cyclohexane-1,2-dicarboxylic acid, isododecyl isohexyl ester of cyclohexane-1,2-dicarboxylic acid, n-tridecyl isohexyl ester of cyclohexane-1,2-dicarboxylic acid, isotridecyl isohexyl ester of cyclohexane-1,2-dicarboxylic acid, isoheptyl n-heptyl ester of cyclohexane-1,2-dicarboxylic acid, n-octyl n-heptyl ester of cyclohexane-1,2-dicarboxylic acid, isooctyl n-heptyl ester of cyclohexane-1,2-dicarboxylic acid, 2-ethylhexyl n-heptyl ester of cyclohexane-1,2-dicarboxylic acid, n-nonyl n-heptyl ester of cyclohexane-1,2-dicarboxylic acid, isononyl n-heptyl ester of cyclohexane-1,2-dicarboxylic acid, 2-propylheptyl n-heptyl ester of cyclohexane-1,2-dicarboxylic acid, n-decyl n-heptyl ester of cyclohexane-1,2-dicarboxylic acid, isodecyl n-heptyl ester of cyclohexane-1,2-dicarboxylic acid, n-undecyl n-heptyl ester of cyclohexane-1,2-dicarboxylic acid, isoundecyl n-heptyl ester of cyclohexane-1,2-dicarboxylic acid, n-dodecyl n-heptyl ester of cyclohexane-1,2-dicarboxylic acid, isododecyl n-heptyl ester of cyclohexane-1,2-dicarboxylic acid, n-tridecyl n-heptyl ester of cyclohexane-1,2-dicarboxylic acid, isotridecyl n-heptyl ester of cyclohexane-1,2-dicarboxylic acid, n-octyl isoheptyl ester of cyclohexane-1,2-dicarboxylic acid, isooctyl isoheptyl ester of cyclohexane-1,2-dicarboxylic acid, 2-ethylhexyl isoheptyl ester of cyclohexane-1,2-dicarboxylic acid, n-nonyl isoheptyl ester of cyclohexane-1,2-dicarboxylic acid, isononyl isoheptyl ester of cyclohexane-1,2-dicarboxylic acid, 2-propylheptyl isoheptyl ester of cyclohexane-1,2-dicarboxylic acid, n-decyl isoheptyl ester of cyclohexane-1,2-dicarboxylic acid, isodecyl isoheptyl ester of cyclohexane-1,2-dicarboxylic acid, n-undecyl isoheptyl ester of cyclohexane-1,2-dicarboxylic acid, isoundecyl isoheptyl ester of cyclohexane-1,2-dicarboxylic acid, n-dodecyl isoheptyl ester of cyclohexane-1,2-dicarboxylic acid, isododecyl isoheptyl ester of cyclohexane-1,2-dicarboxylic acid, n-tridecyl isoheptyl ester of cyclohexane-1,2-dicarboxylic acid, isotridecyl isoheptyl ester of cyclohexane-1,2-dicarboxylic acid, isooctyl n-octyl ester of cyclohexane-1,2-dicarboxylic acid, 2-ethylhexyl n-octyl ester of cyclohexane-1,2-dicarboxylic acid, n-nonyl n-octyl ester of cyclohexane-1,2-dicarboxylic acid, isononyl n-octyl ester of cyclohexane-1,2-dicarboxylic acid, 2-propylheptyl n-octyl ester of cyclohexane-1,2-dicarboxylic acid, n-decyl n-octyl ester of cyclohexane-1,2-dicarboxylic acid, isodecyl n-octyl ester of cyclohexane-1,2-dicarboxylic acid, n-undecyl n-octyl ester of cyclohexane-1,2-dicarboxylic acid, isoundecyl n-octyl ester of cyclohexane-1,2-dicarboxylic acid, n-dodecyl n-octyl ester of cyclohexane-1,2-dicarboxylic acid, isododecyl n-octyl ester of cyclohexane-1,2-dicarboxylic acid, n-tridecyl n-octyl ester of cyclohexane-1,2-dicarboxylic acid, isotridecyl n-octyl ester of cyclohexane-1,2-dicarboxylic acid, 2-ethylhexyl isooctyl ester of cyclohexane-1,2-dicarboxylic acid, n-nonyl isooctyl ester of cyclohexane-1,2-dicarboxylic acid, isononyl isooctyl ester of cyclohexane-1,2-dicarboxylic acid, 2-propylheptyl isooctyl ester of cyclohexane-1,2-dicarboxylic acid, n-decyl isooctyl ester of cyclohexane-1,2-dicarboxylic acid, isodecyl isooctyl ester of cyclohexane-1,2-dicarboxylic acid, n-undecyl isooctyl ester of cyclohexane-1,2-dicarboxylic acid, isoundecyl isooctyl ester of cyclohexane-1,2-dicarboxylic acid, n-dodecyl isooctyl ester of cyclohexane-1,2-dicarboxylic acid, isododecyl isooctyl ester of cyclohexane-1,2-dicarboxylic acid, n-tridecyl isooctyl ester of cyclohexane-1,2-dicarboxylic acid, isotridecyl isooctyl ester of cyclohexane-1,2-dicarboxylic acid, n-nonyl 2-ethylhexyl ester of cyclohexane-1,2-dicarboxylic acid, isononyl 2-ethylhexyl ester of cyclohexane-1,2-dicarboxylic acid, 2-propylheptyl 2-ethylhexyl ester of cyclohexane-1,2-dicarboxylic acid, n-decyl 2-ethylhexyl ester of cyclohexane-1,2-dicarboxylic acid, isodecyl 2-ethylhexyl ester of cyclohexane-1,2-dicarboxylic acid, n-undecyl 2-ethylhexyl ester of cyclohexane-1,2-dicarboxylic acid, isoundecyl 2-ethylhexyl ester of cyclohexane-1,2-dicarboxylic acid, n-dodecyl 2-ethylhexyl ester of cyclohexane-1,2-dicarboxylic acid, isododecyl 2-ethylhexyl ester of cyclohexane-1,2-dicarboxylic acid, n-tridecyl 2-ethylhexyl ester of cyclohexane-1,2-dicarboxylic acid, isotridecyl 2-ethylhexyl ester of cyclohexane-1,2-dicarboxylic acid, isononyl n-nonyl ester of cyclohexane-1,2-dicarboxylic acid, 2-propylheptyl n-nonyl ester of cyclohexane-1,2-dicarboxylic acid, n-decyl n-nonyl ester of cyclohexane-1,2-dicarboxylic acid, isodecyl n-nonyl ester of cyclohexane-1,2-dicarboxylic acid, n-undecyl n-nonyl ester of cyclohexane-1,2-dicarboxylic acid, isoundecyl n-nonyl ester of cyclohexane-1,2-dicarboxylic acid, n-dodecyl n-nonyl ester of cyclohexane-1,2-dicarboxylic acid, isododecyl n-nonyl ester of cyclohexane-1,2-dicarboxylic acid, n-tridecyl n-nonyl ester of cyclohexane-1,2-dicarboxylic acid, isotridecyl n-nonyl ester of cyclohexane-1,2-dicarboxylic acid, 2-propylheptyl isononyl ester of cyclohexane-1,2-dicarboxylic acid, n-decyl isononyl ester of cyclohexane-1,2-dicarboxylic acid, isodecyl isononyl ester of cyclohexane-1,2-dicarboxylic acid, n-undecyl isononyl ester of cyclohexane-1,2-dicarboxylic acid, isoundecyl isononyl ester of cyclohexane-1,2-dicarboxylic acid, n-dodecyl isononyl ester of cyclohexane-1,2-dicarboxylic acid, isododecyl isononyl ester of cyclohexane-1,2-dicarboxylic acid, n-tridecyl isononyl ester of cyclohexane-1,2-dicarboxylic acid, isotridecyl isononyl ester of cyclohexane-1,2-dicarboxylic acid, n-decyl 2-propylheptyl ester of cyclohexane-1,2-dicarboxylic acid, isodecyl 2-propylheptyl ester of cyclohexane-1,2-dicarboxylic acid, n-undecyl 2-propylheptyl ester of cyclohexane-1,2-dicarboxylic acid, isoundecyl 2-propylheptyl ester of cyclohexane-1,2-dicarboxylic acid, n-dodecyl 2-propylheptyl ester of cyclohexane-1,2-dicarboxylic acid, isododecyl 2-propylheptyl ester of cyclohexane-1,2-dicarboxylic acid, n-tridecyl 2-propylheptyl ester of cyclohexane-1,2-dicarboxylic acid, isotridecyl 2-propylheptyl ester of cyclohexane-1,2-dicarboxylic acid, isodecyl n-decyl ester of cyclohexane-1,2-dicarboxylic acid, n-undecyl n-decyl ester of cyclohexane-1,2-dicarboxylic acid, isoundecyl n-decyl ester of cyclohexane-1,2-dicarboxylic acid, n-dodecyl n-decyl ester of cyclohexane-1,2-dicarboxylic acid, isododecyl n-decyl ester of cyclohexane-1,2-dicarboxylic acid, n-tridecyl n-decyl ester of cyclohexane-1,2-dicarboxylic acid, isotridecyl n-decyl ester of cyclohexane-1,2-dicarboxylic acid, n-undecyl isodecyl ester of cyclohexane-1,2-dicarboxylic acid, isoundecyl isodecyl ester of cyclohexane-1,2-dicarboxylic acid, n-dodecyl isodecyl ester of cyclohexane-1,2-dicarboxylic acid, isododecyl isodecyl ester of cyclohexane-1,2-dicarboxylic acid, n-tridecyl isodecyl ester of cyclohexane-1,2-dicarboxylic acid, isotridecyl isodecyl ester of cyclohexane-1,2-dicarboxylic acid, isoundecyl n-undecyl ester of cyclohexane-1,2-dicarboxylic acid, n-dodecyl n-undecyl ester of cyclohexane-1,2-dicarboxylic acid, isododecyl n-undecyl ester of cyclohexane-1,2-dicarboxylic acid, n-tridecyl n-undecyl ester of cyclohexane-1,2-dicarboxylic acid, isotridecyl n-undecyl ester of cyclohexane-1,2-dicarboxylic acid, n-dodecyl isoundecyl ester of cyclohexane-1,2-dicarboxylic acid, isododecyl isoundecyl ester of cyclohexane-1,2-dicarboxylic acid, n-tridecyl isoundecyl ester of cyclohexane-1,2-dicarboxylic acid, isotridecyl isoundecyl ester of cyclohexane-1,2-dicarboxylic acid, isododecyl n-dodecyl ester of cyclohexane-1,2-dicarboxylic acid, n-tridecyl n-dodecyl ester of cyclohexane-1,2-dicarboxylic acid, isotridecyl n-dodecyl ester of cyclohexane-1,2-dicarboxylic acid, n-tridecyl isododecyl ester of cyclohexane-1,2-dicarboxylic acid, isotridecyl isododecyl ester of cyclohexane-1,2-dicarboxylic acid, isotridecyl n-tridecyl ester of cyclohexane-1,2-dicarboxylic acid, methyl cyclohexyl ester of cyclohexane-1,2-dicarboxylic acid, ethyl cyclohexyl ester of cyclohexane-1,2-dicarboxylic acid, n-propyl cyclohexyl ester of cyclohexane-1,2-dicarboxylic acid, isopropyl cyclohexyl ester of cyclohexane-1,2-dicarboxylic acid, n-butyl cyclohexyl ester of cyclohexane-1,2-dicarboxylic acid, tert-butyl cyclohexyl ester of cyclohexane-1,2-dicarboxylic acid, isobutyl cyclohexyl ester of cyclohexane-1,2-dicarboxylic acid, glycol cyclohexyl ester of cyclohexane-1,2-dicarboxylic acid, n-hexyl cyclohexyl ester of cyclohexane-1,2-dicarboxylic acid, isohexyl cyclohexyl ester of cyclohexane-1,2-dicarboxylic acid, n-heptyl cyclohexyl ester of cyclohexane-1,2-dicarboxylic acid, isoheptyl cyclohexyl ester of cyclohexane-1,2-dicarboxylic acid, n-octyl cyclohexyl ester of cyclohexane-1,2-dicarboxylic acid, isooctyl cyclohexyl ester of cyclohexane-1,2-dicarboxylic acid, 2-ethylhexyl cyclohexyl ester of cyclohexane-1,2-dicarboxylic acid, n-nonyl cyclohexyl ester of cyclohexane-1,2-dicarboxylic acid, isononyl cyclohexyl ester of cyclohexane-1,2-dicarboxylic acid, 2-propylheptyl cyclohexyl ester of cyclohexane-1,2-dicarboxylic acid, n-decyl cyclohexyl ester of cyclohexane-1,2-dicarboxylic acid, isodecyl cyclohexyl ester of cyclohexane-1,2-dicarboxylic acid, n-undecyl cyclohexyl ester of cyclohexane-1,2-dicarboxylic acid, isoundecyl cyclohexyl ester of cyclohexane-1,2-dicarboxylic acid, n-dodecyl cyclohexyl ester of cyclohexane-1,2-dicarboxylic acid, isododecyl cyclohexyl ester of cyclohexane-1,2-dicarboxylic acid, n-tridecyl cyclohexyl ester of cyclohexane-1,2-dicarboxylic acid, isotridecyl cyclohexyl ester of cyclohexane-1,2-dicarboxylic acid, methyl isopentyl ester of cyclohexane-1,2-dicarboxylic acid, ethyl isopentyl ester of cyclohexane-1,2-dicarboxylic acid, n-propyl isopentyl ester of cyclohexane-1,2-dicarboxylic acid, isopropyl isopentyl ester of cyclohexane-1,2-dicarboxylic acid, n-butyl isopentyl ester of cyclohexane-1,2-dicarboxylic acid, tert-butyl isopentyl ester of cyclohexane-1,2-dicarboxylic acid, isobutyl isopentyl ester of cyclohexane-1,2-dicarboxylic acid, glycol isopentyl ester of cyclohexane-1,2-dicarboxylic acid, n-hexyl isopentyl ester of cyclohexane-1,2-dicarboxylic acid, isohexyl isopentyl ester of cyclohexane-1,2-dicarboxylic acid, n-heptyl isopentyl ester of cyclohexane-1,2-dicarboxylic acid, isoheptyl isopentyl ester of cyclohexane-1,2-dicarboxylic acid, n-octyl isopentyl ester of cyclohexane-1,2-dicarboxylic acid, isooctyl isopentyl ester of cyclohexane-1,2-dicarboxylic acid, 2-ethylhexyl isopentyl ester of cyclohexane-1,2-dicarboxylic acid, n-nonyl isopentyl ester of cyclohexane-1,2-dicarboxylic acid, isononyl isopentyl ester of cyclohexane-1,2-dicarboxylic acid, 2-propylheptyl isopentyl ester of cyclohexane-1,2-dicarboxylic acid, n-decyl isopentyl ester of cyclohexane-1,2-dicarboxylic acid, isodecyl isopentyl ester of cyclohexane-1,2-dicarboxylic acid, n-undecyl isopentyl ester of cyclohexane-1,2-dicarboxylic acid, isoundecyl isopentyl ester of cyclohexane-1,2-dicarboxylic acid, n-dodecyl isopentyl ester of cyclohexane-1,2-dicarboxylic acid, isododecyl isopentyl ester of cyclohexane-1,2-dicarboxylic acid, n-tridecyl isopentyl ester of cyclohexane-1,2-dicarboxylic acid, isotridecyl isopentyl ester of cyclohexane-1,2-dicarboxylic acid, methyl n-pentyl ester of cyclohexane-1,2-dicarboxylic acid, ethyl n-pentyl ester of cyclohexane-1,2-dicarboxylic acid, n-propyl n-pentyl ester of cyclohexane-1,2-dicarboxylic acid, isopropyl n-pentyl ester of cyclohexane-1,2-dicarboxylic acid, n-butyl n-pentyl ester of cyclohexane-1,2-dicarboxylic acid, tert-butyl n-pentyl ester of cyclohexane-1,2-dicarboxylic acid, isobutyl n-pentyl ester of cyclohexane-1,2-dicarboxylic acid, glycol n-pentyl ester of cyclohexane-1,2-dicarboxylic acid, n-hexyl n-pentyl ester of cyclohexane-1,2-dicarboxylic acid, isohexyl n-pentyl ester of cyclohexane-1,2-dicarboxylic acid, n-heptyl n-pentyl ester of cyclohexane-1,2-dicarboxylic acid, isoheptyl n-pentyl ester of cyclohexane-1,2-dicarboxylic acid, n-octyl n-pentyl ester of cyclohexane-1,2-dicarboxylic acid, isooctyl n-pentyl ester of cyclohexane-1,2-dicarboxylic acid, 2-ethylhexyl n-pentyl ester of cyclohexane-1,2-dicarboxylic acid, n-nonyl n-pentyl ester of cyclohexane-1,2-dicarboxylic acid, isononyl n-pentyl ester of cyclohexane-1,2-dicarboxylic acid, 2-propylheptyl n-pentyl ester of cyclohexane-1,2-dicarboxylic acid, n-decyl n-pentyl ester of cyclohexane-1,2-dicarboxylic acid, isodecyl n-pentyl ester of cyclohexane-1,2-dicarboxylic acid, n-undecyl n-pentyl ester of cyclohexane-1,2-dicarboxylic acid, isoundecyl n-pentyl ester of cyclohexane-1,2-dicarboxylic acid, n-dodecyl n-pentyl ester of cyclohexane-1,2-dicarboxylic acid, isododecyl n-pentyl ester of cyclohexane-1,2-dicarboxylic acid, n-tridecyl n-pentyl ester of cyclohexane-1,2-dicarboxylic acid, isotridecyl n-pentyl ester of cyclohexane-1,2-dicarboxylic acid;

mixed esters of cyclohexane-1,3-dicarboxylic acid with C₁-C₁₃ alcohols, such as, for example, ethyl methyl ester of cyclohexane-1,3-dicarboxylic acid, n-propyl methyl ester of cyclohexane-1,3-dicarboxylic acid, isopropyl methyl ester of cyclohexane-1,3-dicarboxylic acid, n-butyl methyl ester of cyclohexane-1,3-dicarboxylic acid, tert-butyl methyl ester of cyclohexane-1,3-dicarboxylic acid, isobutyl methyl ester of cyclohexane-1,3-dicarboxylic acid, glycol methyl ester of cyclohexane-1,3-dicarboxylic acid, n-hexyl methyl ester of cyclohexane-1,3-dicarboxylic acid, isohexyl methyl ester of cyclohexane-1,3-dicarboxylic acid, n-heptyl methyl ester of cyclohexane-1,3-dicarboxylic acid, isoheptyl methyl ester of cyclohexane-1,3-dicarboxylic acid, n-octyl methyl ester of cyclohexane-1,3-dicarboxylic acid, isooctyl methyl ester of cyclohexane-1,3-dicarboxylic acid, 2-ethylhexyl methyl ester of cyclohexane-1,3-dicarboxylic acid, n-nonyl methyl ester of cyclohexane-1,3-dicarboxylic acid, isononyl methyl ester of cyclohexane-1,3-dicarboxylic acid, 2-propylheptyl methyl ester of cyclohexane-1,3-dicarboxylic acid, n-decyl methyl ester of cyclohexane-1,3-dicarboxylic acid, isodecyl methyl ester of cyclohexane-1,3-dicarboxylic acid, n-undecyl methyl ester of cyclohexane-1,3-dicarboxylic acid, isoundecyl methyl ester of cyclohexane-1,3-dicarboxylic acid, n-dodecyl methyl ester of cyclohexane-1,3-dicarboxylic acid, isododecyl methyl ester of cyclohexane-1,3-dicarboxylic acid, n-tridecyl methyl ester of cyclohexane-1,3-dicarboxylic acid, isotridecyl methyl ester of cyclohexane-1,3-dicarboxylic acid, n-propyl ethyl ester of cyclohexane-1,3-dicarboxylic acid, isopropyl ethyl ester of cyclohexane-1,3-dicarboxylic acid, n-butyl ethyl ester of cyclohexane-1,3-dicarboxylic acid, tert-butyl ethyl ester of cyclohexane-1,3-dicarboxylic acid, isobutyl ethyl ester of cyclohexane-1,3-dicarboxylic acid, glycol ethyl ester of cyclohexane-1,3-dicarboxylic acid, n-hexyl ethyl ester of cyclohexane-1,3-dicarboxylic acid, isohexyl ethyl ester of cyclohexane-1,3-dicarboxylic acid, n-heptyl ethyl ester of cyclohexane-1,3-dicarboxylic acid, isoheptyl ethyl ester of cyclohexane-1,3-dicarboxylic acid, n-octyl ethyl ester of cyclohexane-1,3-dicarboxylic acid, isooctyl ethyl ester of cyclohexane-1,3-dicarboxylic acid, 2-ethylhexyl ethyl ester of cyclohexane-1,3-dicarboxylic acid, n-nonyl ethyl ester of cyclohexane-1,3-dicarboxylic acid, isononyl ethyl ester of cyclohexane-1,3-dicarboxylic acid, 2-propylheptyl ethyl ester of cyclohexane-1,3-dicarboxylic acid, n-decyl ethyl ester of cyclohexane-1,3-dicarboxylic acid, isodecyl ethyl ester of cyclohexane-1,3-dicarboxylic acid, n-undecyl ethyl ester of cyclohexane-1,3-dicarboxylic acid, isoundecyl ethyl ester of cyclohexane-1,3-dicarboxylic acid, n-dodecyl ethyl ester of cyclohexane-1,3-dicarboxylic acid, isododecyl ethyl ester of cyclohexane-1,3-dicarboxylic acid, n-tridecyl ethyl ester of cyclohexane-1,3-dicarboxylic acid, isotridecyl ethyl ester of cyclohexane-1,3-dicarboxylic acid, isopropyl n-propyl ester of cyclohexane-1,3-dicarboxylic acid, n-butyl n-propyl ester of cyclohexane-1,3-dicarboxylic acid, tert-butyl n-propyl ester of cyclohexane-1,3-dicarboxylic acid, isobutyl n-propyl ester of cyclohexane-1,3-dicarboxylic acid, glycol n-propyl ester of cyclohexane-1,3-dicarboxylic acid, n-hexyl n-propyl ester of cyclohexane-1,3-dicarboxylic acid, isohexyl n-propyl ester of cyclohexane-1,3-dicarboxylic acid, n-heptyl n-propyl ester of cyclohexane-1,3-dicarboxylic acid, isoheptyl n-propyl ester of cyclohexane-1,3-dicarboxylic acid, n-octyl n-propyl ester of cyclohexane-1,3-dicarboxylic acid, isooctyl n-propyl ester of cyclohexane-1,3-dicarboxylic acid, 2-ethylhexyl n-propyl ester of cyclohexane-1,3-dicarboxylic acid, n-nonyl n-propyl ester of cyclohexane-1,3-dicarboxylic acid, isononyl n-propyl ester of cyclohexane-1,3-dicarboxylic acid, 2-propylheptyl n-propyl ester of cyclohexane-1,3-dicarboxylic acid, n-decyl n-propyl ester of cyclohexane-1,3-dicarboxylic acid, isodecyl n-propyl ester of cyclohexane-1,3-dicarboxylic acid, n-undecyl n-propyl ester of cyclohexane-1,3-dicarboxylic acid, isoundecyl n-propyl ester of cyclohexane-1,3-dicarboxylic acid, n-dodecyl n-propyl ester of cyclohexane-1,3-dicarboxylic acid, isododecyl n-propyl ester of cyclohexane-1,3-dicarboxylic acid, n-tridecyl n-propyl ester of cyclohexane-1,3-dicarboxylic acid, isotridecyl n-propyl ester of cyclohexane-1,3-dicarboxylic acid, n-butyl isopropyl ester of cyclohexane-1,3-dicarboxylic acid, tert-butyl isopropyl ester of cyclohexane-1,3-dicarboxylic acid, isobutyl isopropyl ester of cyclohexane-1,3-dicarboxylic acid, glycol isopropyl ester of cyclohexane-1,3-dicarboxylic acid, n-hexyl isopropyl ester of cyclohexane-1,3-dicarboxylic acid, isohexyl isopropyl ester of cyclohexane-1,3-dicarboxylic acid, n-heptyl isopropyl ester of cyclohexane-1,3-dicarboxylic acid, isoheptyl isopropyl ester of cyclohexane-1,3-dicarboxylic acid, n-octyl isopropyl ester of cyclohexane-1,3-dicarboxylic acid, isooctyl isopropyl ester of cyclohexane-1,3-dicarboxylic acid, 2-ethylhexyl isopropyl ester of cyclohexane-1,3-dicarboxylic acid, n-nonyl isopropyl ester of cyclohexane-1,3-dicarboxylic acid, isononyl isopropyl ester of cyclohexane-1,3-dicarboxylic acid, 2-propylheptyl isopropyl ester of cyclohexane-1,3-dicarboxylic acid, n-decyl isopropyl ester of cyclohexane-1,3-dicarboxylic acid, isodecyl isopropyl ester of cyclohexane-1,3-dicarboxylic acid, n-undecyl isopropyl ester of cyclohexane-1,3-dicarboxylic acid, isoundecyl isopropyl ester of cyclohexane-1,3-dicarboxylic acid, n-dodecyl isopropyl ester of cyclohexane-1,3-dicarboxylic acid, isododecyl isopropyl ester of cyclohexane-1,3-dicarboxylic acid, n-tridecyl isopropyl ester of cyclohexane-1,3-dicarboxylic acid, isotridecyl isopropyl ester of cyclohexane-1,3-dicarboxylic acid, tert-butyl n-butyl ester of cyclohexane-1,3-dicarboxylic acid, isobutyl n-butyl ester of cyclohexane-1,3-dicarboxylic acid, glycol n-butyl ester of cyclohexane-1,3-dicarboxylic acid, n-hexyl n-butyl ester of cyclohexane-1,3-dicarboxylic acid, isohexyl n-butyl ester of cyclohexane-1,3-dicarboxylic acid, n-heptyl n-butyl ester of cyclohexane-1,3-dicarboxylic acid, isoheptyl n-butyl ester of cyclohexane-1,3-dicarboxylic acid, n-octyl n-butyl ester of cyclohexane-1,3-dicarboxylic acid, isooctyl n-butyl ester of cyclohexane-1,3-dicarboxylic acid, 2-ethylhexyl n-butyl ester of cyclohexane-1,3-dicarboxylic acid, n-nonyl n-butyl ester of cyclohexane-1,3-dicarboxylic acid, isononyl n-butyl ester of cyclohexane-1,3-dicarboxylic acid, 2-propylheptyl n-butyl ester of cyclohexane-1,3-dicarboxylic acid, n-decyl n-butyl ester of cyclohexane-1,3-dicarboxylic acid, isodecyl n-butyl ester of cyclohexane-1,3-dicarboxylic acid, n-undecyl n-butyl ester of cyclohexane-1,3-dicarboxylic acid, isoundecyl n-butyl ester of cyclohexane-1,3-dicarboxylic acid, n-dodecyl n-butyl ester of cyclohexane-1,3-dicarboxylic acid, isododecyl n-butyl ester of cyclohexane-1,3-dicarboxylic acid, n-tridecyl n-butyl ester of cyclohexane-1,3-dicarboxylic acid, isotridecyl n-butyl ester of cyclohexane-1,3-dicarboxylic acid, isobutyl tert-butyl ester of cyclohexane-1,3-dicarboxylic acid, glycol tert-butyl ester of cyclohexane-1,3-dicarboxylic acid, n-hexyl tert-butyl ester of cyclohexane-1,3-dicarboxylic acid, isohexyl tert-butyl ester of cyclohexane-1,3-dicarboxylic acid, n-heptyl tert-butyl ester of cyclohexane-1,3-dicarboxylic acid, isoheptyl tert-butyl ester of cyclohexane-1,3-dicarboxylic acid, n-octyl tert-butyl ester of cyclohexane-1,3-dicarboxylic acid, isooctyl tert-butyl ester of cyclohexane-1,3-dicarboxylic acid, 2-ethylhexyl tert-butyl ester of cyclohexane-1,3-dicarboxylic acid, n-nonyl tert-butyl ester of cyclohexane-1,3-dicarboxylic acid, isononyl tert-butyl ester of cyclohexane-1,3-dicarboxylic acid, 2-propylheptyl tert-butyl ester of cyclohexane-1,3-dicarboxylic acid, n-decyl tert-butyl ester of cyclohexane-1,3-dicarboxylic acid, isodecyl tert-butyl ester of cyclohexane-1,3-dicarboxylic acid, n-undecyl tert-butyl ester of cyclohexane-1,3-dicarboxylic acid, isoundecyl tert-butyl ester of cyclohexane-1,3-dicarboxylic acid, n-dodecyl tert-butyl ester of cyclohexane-1,3-dicarboxylic acid, isododecyl tert-butyl ester of cyclohexane-1,3-dicarboxylic acid, n-tridecyl tert-butyl ester of cyclohexane-1,3-dicarboxylic acid, isotridecyl tert-butyl ester of cyclohexane-1,3-dicarboxylic acid, glycol isobutyl ester of cyclohexane-1,3-dicarboxylic acid, n-hexyl isobutyl ester of cyclohexane-1,3-dicarboxylic acid, isohexyl isobutyl ester of cyclohexane-1,3-dicarboxylic acid, n-heptyl isobutyl ester of cyclohexane-1,3-dicarboxylic acid, isoheptyl isobutyl ester of cyclohexane-1,3-dicarboxylic acid, n-octyl isobutyl ester of cyclohexane-1,3-dicarboxylic acid, isooctyl isobutyl ester of cyclohexane-1,3-dicarboxylic acid, 2-ethylhexyl isobutyl ester of cyclohexane-1,3-dicarboxylic acid, n-nonyl isobutyl ester of cyclohexane-1,3-dicarboxylic acid, isononyl isobutyl ester of cyclohexane-1,3-dicarboxylic acid, 2-propylheptyl isobutyl ester of cyclohexane-1,3-dicarboxylic acid, n-decyl isobutyl ester of cyclohexane-1,3-dicarboxylic acid, isodecyl isobutyl ester of cyclohexane-1,3-dicarboxylic acid, n-undecyl isobutyl ester of cyclohexane-1,3-dicarboxylic acid, isoundecyl isobutyl ester of cyclohexane-1,3-dicarboxylic acid, n-dodecyl isobutyl ester of cyclohexane-1,3-dicarboxylic acid, isododecyl isobutyl ester of cyclohexane-1,3-dicarboxylic acid, n-tridecyl isobutyl ester of cyclohexane-1,3-dicarboxylic acid, isotridecyl isobutyl ester of cyclohexane-1,3-dicarboxylic acid, n-hexyl glycol ester of cyclohexane-1,3-dicarboxylic acid, isohexyl glycol ester of cyclohexane-1,3-dicarboxylic acid, n-heptyl glycol ester of cyclohexane-1,3-dicarboxylic acid, isoheptyl glycol ester of cyclohexane-1,3-dicarboxylic acid, n-octyl glycol ester of cyclohexane-1,3-dicarboxylic acid, isooctyl glycol ester of cyclohexane-1,3-dicarboxylic acid, 2-ethylhexyl glycol ester of cyclohexane-1,3-dicarboxylic acid, n-nonyl glycol ester of cyclohexane-1,3-dicarboxylic acid, isononyl glycol ester of cyclohexane-1,3-dicarboxylic acid, 2-propylheptyl glycol ester of cyclohexane-1,3-dicarboxylic acid, n-decyl glycol ester of cyclohexane-1,3-dicarboxylic acid, isodecyl glycol ester of cyclohexane-1,3-dicarboxylic acid, n-undecyl glycol ester of cyclohexane-1,3-dicarboxylic acid, isoundecyl glycol ester of cyclohexane-1,3-dicarboxylic acid, n-dodecyl glycol ester of cyclohexane-1,3-dicarboxylic acid, isododecyl glycol ester of cyclohexane-1,3-dicarboxylic acid, n-tridecyl glycol ester of cyclohexane-1,3-dicarboxylic acid, isotridecyl glycol ester of cyclohexane-1,3-dicarboxylic acid, isohexyl n-hexyl ester of cyclohexane-1,3-dicarboxylic acid, n-heptyl n-hexyl ester of cyclohexane-1,3-dicarboxylic acid, isoheptyl n-hexyl ester of cyclohexane-1,3-dicarboxylic acid, n-octyl n-hexyl ester of cyclohexane-1,3-dicarboxylic acid, isooctyl n-hexyl ester of cyclohexane-1,3-dicarboxylic acid, 2-ethylhexyl n-hexyl ester of cyclohexane-1,3-dicarboxylic acid, n-nonyl n-hexyl ester of cyclohexane-1,3-dicarboxylic acid, isononyl n-hexyl ester of cyclohexane-1,3-dicarboxylic acid, 2-propylheptyl n-hexyl ester of cyclohexane-1,3-dicarboxylic acid, n-decyl n-hexyl ester of cyclohexane-1,3-dicarboxylic acid, isodecyl n-hexyl ester of cyclohexane-1,3-dicarboxylic acid, n-undecyl n-hexyl ester of cyclohexane-1,3-dicarboxylic acid, isoundecyl n-hexyl ester of cyclohexane-1,3-dicarboxylic acid, n-dodecyl n-hexyl ester of cyclohexane-1,3-dicarboxylic acid, isododecyl n-hexyl ester of cyclohexane-1,3-dicarboxylic acid, n-tridecyl n-hexyl ester of cyclohexane-1,3-dicarboxylic acid, isotridecyl n-hexyl ester of cyclohexane-1,3-dicarboxylic acid, n-heptyl isohexyl ester of cyclohexane-1,3-dicarboxylic acid, isoheptyl isohexyl ester of cyclohexane-1,3-dicarboxylic acid, n-octyl isohexyl ester of cyclohexane-1,3-dicarboxylic acid, isooctyl isohexyl ester of cyclohexane-1,3-dicarboxylic acid, 2-ethylhexyl isohexyl ester of cyclohexane-1,3-dicarboxylic acid, n-nonyl isohexyl ester of cyclohexane-1,3-dicarboxylic acid, isononyl isohexyl ester of cyclohexane-1,3-dicarboxylic acid, 2-propylheptyl isohexyl ester of cyclohexane-1,3-dicarboxylic acid, n-decyl isohexyl ester of cyclohexane-1,3-dicarboxylic acid, isodecyl isohexyl ester of cyclohexane-1,3-dicarboxylic acid, n-undecyl isohexyl ester of cyclohexane-1,3-dicarboxylic acid, isoundecyl isohexyl ester of cyclohexane-1,3-dicarboxylic acid, n-dodecyl isohexyl ester of cyclohexane-1,3-dicarboxylic acid, isododecyl isohexyl ester of cyclohexane-1,3-dicarboxylic acid, n-tridecyl isohexyl ester of cyclohexane-1,3-dicarboxylic acid, isotridecyl isohexyl ester of cyclohexane-1,3-dicarboxylic acid, isoheptyl n-heptyl ester of cyclohexane-1,3-dicarboxylic acid, n-octyl n-heptyl ester of cyclohexane-1,3-dicarboxylic acid, isooctyl n-heptyl ester of cyclohexane-1,3-dicarboxylic acid, 2-ethylhexyl n-heptyl ester of cyclohexane-1,3-dicarboxylic acid, n-nonyl n-heptyl ester of cyclohexane-1,3-dicarboxylic acid, isononyl n-heptyl ester of cyclohexane-1,3-dicarboxylic acid, 2-propylheptyl n-heptyl ester of cyclohexane-1,3-dicarboxylic acid, n-decyl n-heptyl ester of cyclohexane-1,3-dicarboxylic acid, isodecyl n-heptyl ester of cyclohexane-1,3-dicarboxylic acid, n-undecyl n-heptyl ester of cyclohexane-1,3-dicarboxylic acid, isoundecyl n-heptyl ester of cyclohexane-1,3-dicarboxylic acid, n-dodecyl n-heptyl ester of cyclohexane-1,3-dicarboxylic acid, isododecyl n-heptyl ester of cyclohexane-1,3-dicarboxylic acid, n-tridecyl n-heptyl ester of cyclohexane-1,3-dicarboxylic acid, isotridecyl n-heptyl ester of cyclohexane-1,3-dicarboxylic acid, n-octyl isoheptyl ester of cyclohexane-1,3-dicarboxylic acid, isooctyl isoheptyl ester of cyclohexane-1,3-dicarboxylic acid, 2-ethylhexyl isoheptyl ester of cyclohexane-1,3-dicarboxylic acid, n-nonyl isoheptyl ester of cyclohexane-1,3-dicarboxylic acid, isononyl isoheptyl ester of cyclohexane-1,3-dicarboxylic acid, 2-propylheptyl isoheptyl ester of cyclohexane-1,3-dicarboxylic acid, n-decyl isoheptyl ester of cyclohexane-1,3-dicarboxylic acid, isodecyl isoheptyl ester of cyclohexane-1,3-dicarboxylic acid, n-undecyl isoheptyl ester of cyclohexane-1,3-dicarboxylic acid, isoundecyl isoheptyl ester of cyclohexane-1,3-dicarboxylic acid, n-dodecyl isoheptyl ester of cyclohexane-1,3-dicarboxylic acid, isododecyl isoheptyl ester of cyclohexane-1,3-dicarboxylic acid, n-tridecyl isoheptyl ester of cyclohexane-1,3-dicarboxylic acid, isotridecyl isoheptyl ester of cyclohexane-1,3-dicarboxylic acid, isooctyl n-octyl ester of cyclohexane-1,3-dicarboxylic acid, 2-ethylhexyl n-octyl ester of cyclohexane-1,3-dicarboxylic acid, n-nonyl n-octyl ester of cyclohexane-1,3-dicarboxylic acid, isononyl n-octyl ester of cyclohexane-1,3-dicarboxylic acid, 2-propylheptyl n-octyl ester of cyclohexane-1,3-dicarboxylic acid, n-decyl n-octyl ester of cyclohexane-1,3-dicarboxylic acid, isodecyl n-octyl ester of cyclohexane-1,3-dicarboxylic acid, n-undecyl n-octyl ester of cyclohexane-1,3-dicarboxylic acid, isoundecyl n-octyl ester of cyclohexane-1,3-dicarboxylic acid, n-dodecyl n-octyl ester of cyclohexane-1,3-dicarboxylic acid, isododecyl n-octyl ester of cyclohexane-1,3-dicarboxylic acid, n-tridecyl n-octyl ester of cyclohexane-1,3-dicarboxylic acid, isotridecyl n-octyl ester of cyclohexane-1,3-dicarboxylic acid, 2-ethylhexyl isooctyl ester of cyclohexane-1,3-dicarboxylic acid, n-nonyl isooctyl ester of cyclohexane-1,3-dicarboxylic acid, isononyl isooctyl ester of cyclohexane-1,3-dicarboxylic acid, 2-propylheptyl isooctyl ester of cyclohexane-1,3-dicarboxylic acid, n-decyl isooctyl ester of cyclohexane-1,3-dicarboxylic acid, isodecyl isooctyl ester of cyclohexane-1,3-dicarboxylic acid, n-undecyl isooctyl ester of cyclohexane-1,3-dicarboxylic acid, isoundecyl isooctyl ester of cyclohexane-1,3-dicarboxylic acid, n-dodecyl isooctyl ester of cyclohexane-1,3-dicarboxylic acid, isododecyl isooctyl ester of cyclohexane-1,3-dicarboxylic acid, n-tridecyl isooctyl ester of cyclohexane-1,3-dicarboxylic acid, isotridecyl isooctyl ester of cyclohexane-1,3-dicarboxylic acid, n-nonyl 2-ethylhexyl ester of cyclohexane-1,3-dicarboxylic acid, isononyl 2-ethylhexyl ester of cyclohexane-1,3-dicarboxylic acid, 2-propylheptyl 2-ethylhexyl ester of cyclohexane-1,3-dicarboxylic acid, n-decyl 2-ethylhexyl ester of cyclohexane-1,3-dicarboxylic acid, isodecyl 2-ethylhexyl ester of cyclohexane-1,3-dicarboxylic acid, n-undecyl 2-ethylhexyl ester of cyclohexane-1,3-dicarboxylic acid, isoundecyl 2-ethylhexyl ester of cyclohexane-1,3-dicarboxylic acid, n-dodecyl 2-ethylhexyl ester of cyclohexane-1,3-dicarboxylic acid, isododecyl 2-ethylhexyl ester of cyclohexane-1,3-dicarboxylic acid, n-tridecyl 2-ethylhexyl ester of cyclohexane-1,3-dicarboxylic acid, isotridecyl 2-ethylhexyl ester of cyclohexane-1,3-dicarboxylic acid, isononyl n-nonyl ester of cyclohexane-1,3-dicarboxylic acid, 2-propylheptyl n-nonyl ester of cyclohexane-1,3-dicarboxylic acid, n-decyl n-nonyl ester of cyclohexane-1,3-dicarboxylic acid, isodecyl n-nonyl ester of cyclohexane-1,3-dicarboxylic acid, n-undecyl n-nonyl ester of cyclohexane-1,3-dicarboxylic acid, isoundecyl n-nonyl ester of cyclohexane-1,3-dicarboxylic acid, n-dodecyl n-nonyl ester of cyclohexane-1,3-dicarboxylic acid, isododecyl n-nonyl ester of cyclohexane-1,3-dicarboxylic acid, n-tridecyl n-nonyl ester of cyclohexane-1,3-dicarboxylic acid, isotridecyl n-nonyl ester of cyclohexane-1,3-dicarboxylic acid, 2-propylheptyl isononyl ester of cyclohexane-1,3-dicarboxylic acid, n-decyl isononyl ester of cyclohexane-1,3-dicarboxylic acid, isodecyl isononyl ester of cyclohexane-1,3-dicarboxylic acid, n-undecyl isononyl ester of cyclohexane-1,3-dicarboxylic acid, isoundecyl isononyl ester of cyclohexane-1,3-dicarboxylic acid, n-dodecyl isononyl ester of cyclohexane-1,3-dicarboxylic acid, isododecyl isononyl ester of cyclohexane-1,3-dicarboxylic acid, n-tridecyl isononyl ester of cyclohexane-1,3-dicarboxylic acid, isotridecyl isononyl ester of cyclohexane-1,3-dicarboxylic acid, n-decyl 2-propylheptyl ester of cyclohexane-1,3-dicarboxylic acid, isodecyl 2-propylheptyl ester of cyclohexane-1,3-dicarboxylic acid, n-undecyl 2-propylheptyl ester of cyclohexane-1,3-dicarboxylic acid, isoundecyl 2-propylheptyl ester of cyclohexane-1,3-dicarboxylic acid, n-dodecyl 2-propylheptyl ester of cyclohexane-1,3-dicarboxylic acid, isododecyl 2-propylheptyl ester of cyclohexane-1,3-dicarboxylic acid, n-tridecyl 2-propylheptyl ester of cyclohexane-1,3-dicarboxylic acid, isotridecyl 2-propylheptyl ester of cyclohexane-1,3-dicarboxylic acid, isodecyl n-decyl ester of cyclohexane-1,3-dicarboxylic acid, n-undecyl n-decyl ester of cyclohexane-1,3-dicarboxylic acid, isoundecyl n-decyl ester of cyclohexane-1,3-dicarboxylic acid, n-dodecyl n-decyl ester of cyclohexane-1,3-dicarboxylic acid, isododecyl n-decyl ester of cyclohexane-1,3-dicarboxylic acid, n-tridecyl n-decyl ester of cyclohexane-1,3-dicarboxylic acid, isotridecyl n-decyl ester of cyclohexane-1,3-dicarboxylic acid, n-undecyl isodecyl ester of cyclohexane-1,3-dicarboxylic acid, isoundecyl isodecyl ester of cyclohexane-1,3-dicarboxylic acid, n-dodecyl isodecyl ester of cyclohexane-1,3-dicarboxylic acid, isododecyl isodecyl ester of cyclohexane-1,3-dicarboxylic acid, n-tridecyl isodecyl ester of cyclohexane-1,3-dicarboxylic acid, isotridecyl isodecyl ester of cyclohexane-1,3-dicarboxylic acid, isoundecyl n-undecyl ester of cyclohexane-1,3-dicarboxylic acid, n-dodecyl n-undecyl ester of cyclohexane-1,3-dicarboxylic acid, isododecyl n-undecyl ester of cyclohexane-1,3-dicarboxylic acid, n-tridecyl n-undecyl ester of cyclohexane-1,3-dicarboxylic acid, isotridecyl n-undecyl ester of cyclohexane-1,3-dicarboxylic acid, n-dodecyl isoundecyl ester of cyclohexane-1,3-dicarboxylic acid, isododecyl isoundecyl ester of cyclohexane-1,3-dicarboxylic acid, n-tridecyl isoundecyl ester of cyclohexane-1,3-dicarboxylic acid, isotridecyl isoundecyl ester of cyclohexane-1,3-dicarboxylic acid, isododecyl n-dodecyl ester of cyclohexane-1,3-dicarboxylic acid, n-tridecyl n-dodecyl ester of cyclohexane-1,3-dicarboxylic acid, isotridecyl n-dodecyl ester of cyclohexane-1,3-dicarboxylic acid, n-tridecyl isododecyl ester of cyclohexane-1,3-dicarboxylic acid, isotridecyl isododecyl ester of cyclohexane-1,3-dicarboxylic acid, isotridecyl n-tridecyl ester of cyclohexane-1,3-dicarboxylic acid, methyl cyclohexyl ester of cyclohexane-1,3-dicarboxylic acid, ethyl cyclohexyl ester of cyclohexane-1,3-dicarboxylic acid, n-propyl cyclohexyl ester of cyclohexane-1,3-dicarboxylic acid, isopropyl cyclohexyl ester of cyclohexane-1,3-dicarboxylic acid, n-butyl cyclohexyl ester of cyclohexane-1,3-dicarboxylic acid, tert-butyl cyclohexyl ester of cyclohexane-1,3-dicarboxylic acid, isobutyl cyclohexyl ester of cyclohexane-1,3-dicarboxylic acid, glycol cyclohexyl ester of cyclohexane-1,3-dicarboxylic acid, n-hexyl cyclohexyl ester of cyclohexane-1,3-dicarboxylic acid, isohexyl cyclohexyl ester of cyclohexane-1,3-dicarboxylic acid, n-heptyl cyclohexyl ester of cyclohexane-1,3-dicarboxylic acid, isoheptyl cyclohexyl ester of cyclohexane-1,3-dicarboxylic acid, n-octyl cyclohexyl ester of cyclohexane-1,3-dicarboxylic acid, isooctyl cyclohexyl ester of cyclohexane-1,3-dicarboxylic acid, 2-ethylhexyl cyclohexyl ester of cyclohexane-1,3-dicarboxylic acid, n-nonyl cyclohexyl ester of cyclohexane-1,3-dicarboxylic acid, isononyl cyclohexyl ester of cyclohexane-1,3-dicarboxylic acid, 2-propylheptyl cyclohexyl ester of cyclohexane-1,3-dicarboxylic acid, n-decyl cyclohexyl ester of cyclohexane-1,3-dicarboxylic acid, isodecyl cyclohexyl ester of cyclohexane-1,3-dicarboxylic acid, n-undecyl cyclohexyl ester of cyclohexane-1,3-dicarboxylic acid, isoundecyl cyclohexyl ester of cyclohexane-1,3-dicarboxylic acid, n-dodecyl cyclohexyl ester of cyclohexane-1,3-dicarboxylic acid, isododecyl cyclohexyl ester of cyclohexane-1,3-dicarboxylic acid, n-tridecyl cyclohexyl ester of cyclohexane-1,3-dicarboxylic acid, isotridecyl cyclohexyl ester of cyclohexane-1,3-dicarboxylic acid, methyl isopentyl ester of cyclohexane-1,3-dicarboxylic acid, ethyl isopentyl ester of cyclohexane-1,3-dicarboxylic acid, n-propyl isopentyl ester of cyclohexane-1,3-dicarboxylic acid, isopropyl isopentyl ester of cyclohexane-1,3-dicarboxylic acid, n-butyl isopentyl ester of cyclohexane-1,3-dicarboxylic acid, tert-butyl isopentyl ester of cyclohexane-1,3-dicarboxylic acid, isobutyl isopentyl ester of cyclohexane-1,3-dicarboxylic acid, glycol isopentyl ester of cyclohexane-1,3-dicarboxylic acid, n-hexyl isopentyl ester of cyclohexane-1,3-dicarboxylic acid, isohexyl isopentyl ester of cyclohexane-1,3-dicarboxylic acid, n-heptyl isopentyl ester of cyclohexane-1,3-dicarboxylic acid, isoheptyl isopentyl ester of cyclohexane-1,3-dicarboxylic acid, n-octyl isopentyl ester of cyclohexane-1,3-dicarboxylic acid, isooctyl isopentyl ester of cyclohexane-1,3-dicarboxylic acid, 2-ethylhexyl isopentyl ester of cyclohexane-1,3-dicarboxylic acid, n-nonyl isopentyl ester of cyclohexane-1,3-dicarboxylic acid, isononyl isopentyl ester of cyclohexane-1,3-dicarboxylic acid, 2-propylheptyl isopentyl ester of cyclohexane-1,3-dicarboxylic acid, n-decyl isopentyl ester of cyclohexane-1,3-dicarboxylic acid, isodecyl isopentyl ester of cyclohexane-1,3-dicarboxylic acid, n-undecyl isopentyl ester of cyclohexane-1,3-dicarboxylic acid, isoundecyl isopentyl ester of cyclohexane-1,3-dicarboxylic acid, n-dodecyl isopentyl ester of cyclohexane-1,3-dicarboxylic acid, isododecyl isopentyl ester of cyclohexane-1,3-dicarboxylic acid, n-tridecyl isopentyl ester of cyclohexane-1,3-dicarboxylic acid, isotridecyl isopentyl ester of cyclohexane-1,3-dicarboxylic acid, methyl n-pentyl ester of cyclohexane-1,3-dicarboxylic acid, ethyl n-pentyl ester of cyclohexane-1,3-dicarboxylic acid, n-propyl n-pentyl ester of cyclohexane-1,3-dicarboxylic acid, isopropyl n-pentyl ester of cyclohexane-1,3-dicarboxylic acid, n-butyl n-pentyl ester of cyclohexane-1,3-dicarboxylic acid, tert-butyl n-pentyl ester of cyclohexane-1,3-dicarboxylic acid, isobutyl n-pentyl ester of cyclohexane-1,3-dicarboxylic acid, glycol n-pentyl ester of cyclohexane-1,3-dicarboxylic acid, n-hexyl n-pentyl ester of cyclohexane-1,3-dicarboxylic acid, isohexyl n-pentyl ester of cyclohexane-1,3-dicarboxylic acid, n-heptyl n-pentyl ester of cyclohexane-1,3-dicarboxylic acid, isoheptyl n-pentyl ester of cyclohexane-1,3-dicarboxylic acid, n-octyl n-pentyl ester of cyclohexane-1,3-dicarboxylic acid, isooctyl n-pentyl ester of cyclohexane-1,3-dicarboxylic acid, 2-ethylhexyl n-pentyl ester of cyclohexane-1,3-dicarboxylic acid, n-nonyl n-pentyl ester of cyclohexane-1,3-dicarboxylic acid, isononyl n-pentyl ester of cyclohexane-1,3-dicarboxylic acid, 2-propylheptyl n-pentyl ester of cyclohexane-1,3-dicarboxylic acid, n-decyl n-pentyl ester of cyclohexane-1,3-dicarboxylic acid, isodecyl n-pentyl ester of cyclohexane-1,3-dicarboxylic acid, n-undecyl n-pentyl ester of cyclohexane-1,3-dicarboxylic acid, isoundecyl n-pentyl ester of cyclohexane-1,3-dicarboxylic acid, n-dodecyl n-pentyl ester of cyclohexane-1,3-dicarboxylic acid, isododecyl n-pentyl ester of cyclohexane-1,3-dicarboxylic acid, n-tridecyl n-pentyl ester of cyclohexane-1,3-dicarboxylic acid, isotridecyl n-pentyl ester of cyclohexane-1,3-dicarboxylic acid;

mixed esters of cyclohexane-1,4-dicarboxylic acid with C₁ to C₁₃ alcohols, such as, for example, ethyl methyl ester of cyclohexane-1,4-dicarboxylic acid, n-propyl methyl ester of cyclohexane-1,4-dicarboxylic acid, isopropyl methyl ester of cyclohexane-1,4-dicarboxylic acid, n-butyl methyl ester of cyclohexane-1,4-dicarboxylic acid, tert-butyl methyl ester of cyclohexane-1,4-dicarboxylic acid, isobutyl methyl ester of cyclohexane-1,4-dicarboxylic acid, glycol methyl ester of cyclohexane-1,4-dicarboxylic acid, n-hexyl methyl ester of cyclohexane-1,4-dicarboxylic acid, isohexyl methyl ester of cyclohexane-1,4-dicarboxylic acid, n-heptyl methyl ester of cyclohexane-1,4-dicarboxylic acid, isoheptyl methyl ester of cyclohexane-1,4-dicarboxylic acid, n-octyl methyl ester of cyclohexane-1,4-dicarboxylic acid, isooctyl methyl ester of cyclohexane-1,4-dicarboxylic acid, 2-ethylhexyl methyl ester of cyclohexane-1,4-dicarboxylic acid, n-nonyl methyl ester of cyclohexane-1,4-dicarboxylic acid, isononyl methyl ester of cyclohexane-1,4-dicarboxylic acid, 2-propylheptyl methyl ester of cyclohexane-1,4-dicarboxylic acid, n-decyl methyl ester of cyclohexane-1,4-dicarboxylic acid, isodecyl methyl ester of cyclohexane-1,4-dicarboxylic acid, n-undecyl methyl ester of cyclohexane-1,4-dicarboxylic acid, isoundecyl methyl ester of cyclohexane-1,4-dicarboxylic acid, n-dodecyl methyl ester of cyclohexane-1,4-dicarboxylic acid, isododecyl methyl ester of cyclohexane-1,4-dicarboxylic acid, n-tridecyl methyl ester of cyclohexane-1,4-dicarboxylic acid, isotridecyl methyl ester of cyclohexane-1,4-dicarboxylic acid, n-propyl ethyl ester of cyclohexane-1,4-dicarboxylic acid, isopropyl ethyl ester of cyclohexane-1,4-dicarboxylic acid, n-butyl ethyl ester of cyclohexane-1,4-dicarboxylic acid, tert-butyl ethyl ester of cyclohexane-1,4-dicarboxylic acid, isobutyl ethyl ester of cyclohexane-1,4-dicarboxylic acid, glycol ethyl ester of cyclohexane-1,4-dicarboxylic acid, n-hexyl ethyl ester of cyclohexane-1,4-dicarboxylic acid, isohexyl ethyl ester of cyclohexane-1,4-dicarboxylic acid, n-heptyl ethyl ester of cyclohexane-1,4-dicarboxylic acid, isoheptyl ethyl ester of cyclohexane-1,4-dicarboxylic acid, n-octyl ethyl ester of cyclohexane-1,4-dicarboxylic acid, isooctyl ethyl ester of cyclohexane-1,4-dicarboxylic acid, 2-ethylhexyl ethyl ester of cyclohexane-1,4-dicarboxylic acid, n-nonyl ethyl ester of cyclohexane-1,4-dicarboxylic acid, isononyl ethyl ester of cyclohexane-1,4-dicarboxylic acid, 2-propylheptyl ethyl ester of cyclohexane-1,4-dicarboxylic acid, n-decyl ethyl ester of cyclohexane-1,4-dicarboxylic acid, isodecyl ethyl ester of cyclohexane-1,4-dicarboxylic acid, n-undecyl ethyl ester of cyclohexane-1,4-dicarboxylic acid, isoundecyl ethyl ester of cyclohexane-1,4-dicarboxylic acid, n-dodecyl ethyl ester of cyclohexane-1,4-dicarboxylic acid, isododecyl ethyl ester of cyclohexane-1,4-dicarboxylic acid, n-tridecyl ethyl ester of cyclohexane-1,4-dicarboxylic acid, isotridecyl ethyl ester of cyclohexane-1,4-dicarboxylic acid, isopropyl n-propyl ester of cyclohexane-1,4-dicarboxylic acid, n-butyl n-propyl ester of cyclohexane-1,4-dicarboxylic acid, tert-butyl n-propyl ester of cyclohexane-1,4-dicarboxylic acid, isobutyl n-propyl ester of cyclohexane-1,4-dicarboxylic acid, glycol n-propyl ester of cyclohexane-1,4-dicarboxylic acid, n-hexyl n-propyl ester of cyclohexane-1,4-dicarboxylic acid, isohexyl n-propyl ester of cyclohexane-1,4-dicarboxylic acid, n-heptyl n-propyl ester of cyclohexane-1,4-dicarboxylic acid, isoheptyl n-propyl ester of cyclohexane-1,4-dicarboxylic acid, n-octyl n-propyl ester of cyclohexane-1,4-dicarboxylic acid, isooctyl n-propyl ester of cyclohexane-1,4-dicarboxylic acid, 2-ethylhexyl n-propyl ester of cyclohexane-1,4-dicarboxylic acid, n-nonyl n-propyl ester of cyclohexane-1,4-dicarboxylic acid, isononyl n-propyl ester of cyclohexane-1,4-dicarboxylic acid, 2-propylheptyl n-propyl ester of cyclohexane-1,4-dicarboxylic acid, n-decyl n-propyl ester of cyclohexane-1,4-dicarboxylic acid, isodecyl n-propyl ester of cyclohexane-1,4-dicarboxylic acid, n-undecyl n-propyl ester of cyclohexane-1,4-dicarboxylic acid, isoundecyl n-propyl ester of cyclohexane-1,4-dicarboxylic acid, n-dodecyl n-propyl ester of cyclohexane-1,4-dicarboxylic acid, isododecyl n-propyl ester of cyclohexane-1,4-dicarboxylic acid, n-tridecyl n-propyl ester of cyclohexane-1,4-dicarboxylic acid, isotridecyl n-propyl ester of cyclohexane-1,4-dicarboxylic acid, n-butyl isopropyl ester of cyclohexane-1,4-dicarboxylic acid, tert-butyl isopropyl ester of cyclohexane-1,4-dicarboxylic acid, isobutyl isopropyl ester of cyclohexane-1,4-dicarboxylic acid, glycol isopropyl ester of cyclohexane-1,4-dicarboxylic acid, n-hexyl isopropyl ester of cyclohexane-1,4-dicarboxylic acid, isohexyl isopropyl ester of cyclohexane-1,4-dicarboxylic acid, n-heptyl isopropyl ester of cyclohexane-1,4-dicarboxylic acid, isoheptyl isopropyl ester of cyclohexane-1,4-dicarboxylic acid, n-octyl isopropyl ester of cyclohexane-1,4-dicarboxylic acid, isooctyl isopropyl ester of cyclohexane-1,4-dicarboxylic acid, 2-ethylhexyl isopropyl ester of cyclohexane-1,4-dicarboxylic acid, n-nonyl isopropyl ester of cyclohexane-1,4-dicarboxylic acid, isononyl isopropyl ester of cyclohexane-1,4-dicarboxylic acid, 2-propylheptyl isopropyl ester of cyclohexane-1,4-dicarboxylic acid, n-decyl isopropyl ester of cyclohexane-1,4-dicarboxylic acid, isodecyl isopropyl ester of cyclohexane-1,4-dicarboxylic acid, n-undecyl isopropyl ester of cyclohexane-1,4-dicarboxylic acid, isoundecyl isopropyl ester of cyclohexane-1,4-dicarboxylic acid, n-dodecyl isopropyl ester of cyclohexane-1,4-dicarboxylic acid, isododecyl isopropyl ester of cyclohexane-1,4-dicarboxylic acid, n-tridecyl isopropyl ester of cyclohexane-1,4-dicarboxylic acid, isotridecyl isopropyl ester of cyclohexane-1,4-dicarboxylic acid, tert-butyl n-butyl ester of cyclohexane-1,4-dicarboxylic acid, isobutyl n-butyl ester of cyclohexane-1,4-dicarboxylic acid, glycol n-butyl ester of cyclohexane-1,4-dicarboxylic acid, n-hexyl n-butyl ester of cyclohexane-1,4-dicarboxylic acid, isohexyl n-butyl ester of cyclohexane-1,4-dicarboxylic acid, n-heptyl n-butyl ester of cyclohexane-1,4-dicarboxylic acid, isoheptyl n-butyl ester of cyclohexane-1,4-dicarboxylic acid, n-octyl n-butyl ester of cyclohexane-1,4-dicarboxylic acid, isooctyl n-butyl ester of cyclohexane-1,4-dicarboxylic acid, 2-ethylhexyl n-butyl ester of cyclohexane-1,4-dicarboxylic acid, n-nonyl n-butyl ester of cyclohexane-1,4-dicarboxylic acid, isononyl n-butyl ester of cyclohexane-1,4-dicarboxylic acid, 2-propylheptyl n-butyl ester of cyclohexane-1,4-dicarboxylic acid, n-decyl n-butyl ester of cyclohexane-1,4-dicarboxylic acid, isodecyl n-butyl ester of cyclohexane-1,4-dicarboxylic acid, n-undecyl n-butyl ester of cyclohexane-1,4-dicarboxylic acid, isoundecyl n-butyl ester of cyclohexane-1,4-dicarboxylic acid, n-dodecyl n-butyl ester of cyclohexane-1,4-dicarboxylic acid, isododecyl n-butyl ester of cyclohexane-1,4-dicarboxylic acid, n-tridecyl n-butyl ester of cyclohexane-1,4-dicarboxylic acid, isotridecyl n-butyl ester of cyclohexane-1,4-dicarboxylic acid, isobutyl tert-butyl ester of cyclohexane-1,4-dicarboxylic acid, glycol tert-butyl ester of cyclohexane-1,4-dicarboxylic acid, n-hexyl tert-butyl ester of cyclohexane-1,4-dicarboxylic acid, isohexyl tert-butyl ester of cyclohexane-1,4-dicarboxylic acid, n-heptyl tert-butyl ester of cyclohexane-1,4-dicarboxylic acid, isoheptyl tert-butyl ester of cyclohexane-1,4-dicarboxylic acid, n-octyl tert-butyl ester of cyclohexane-1,4-dicarboxylic acid, isooctyl tert-butyl ester of cyclohexane-1,4-dicarboxylic acid, 2-ethylhexyl tert-butyl ester of cyclohexane-1,4-dicarboxylic acid, n-nonyl tert-butyl ester of cyclohexane-1,4-dicarboxylic acid, isononyl tert-butyl ester of cyclohexane-1,4-dicarboxylic acid, 2-propylheptyl tert-butyl ester of cyclohexane-1,4-dicarboxylic acid, n-decyl tert-butyl ester of cyclohexane-1,4-dicarboxylic acid, isodecyl tert-butyl ester of cyclohexane-1,4-dicarboxylic acid, n-undecyl tert-butyl ester of cyclohexane-1,4-dicarboxylic acid, isoundecyl tert-butyl ester of cyclohexane-1,4-dicarboxylic acid, n-dodecyl tert-butyl ester of cyclohexane-1,4-dicarboxylic acid, isododecyl tert-butyl ester of cyclohexane-1,4-dicarboxylic acid, n-tridecyl tert-butyl ester of cyclohexane-1,4-dicarboxylic acid, isotridecyl tert-butyl ester of cyclohexane-1,4-dicarboxylic acid, glycol isobutyl ester of cyclohexane-1,4-dicarboxylic acid, n-hexyl isobutyl ester of cyclohexane-1,4-dicarboxylic acid, isohexyl isobutyl ester of cyclohexane-1,4-dicarboxylic acid, n-heptyl isobutyl ester of cyclohexane-1,4-dicarboxylic acid, isoheptyl isobutyl ester of cyclohexane-1,4-dicarboxylic acid, n-octyl isobutyl ester of cyclohexane-1,4-dicarboxylic acid, isooctyl isobutyl ester of cyclohexane-1,4-dicarboxylic acid, 2-ethylhexyl isobutyl ester of cyclohexane-1,4-dicarboxylic acid, n-nonyl isobutyl ester of cyclohexane-1,4-dicarboxylic acid, isononyl isobutyl ester of cyclohexane-1,4-dicarboxylic acid, 2-propylheptyl isobutyl ester of cyclohexane-1,4-dicarboxylic acid, n-decyl isobutyl ester of cyclohexane-1,4-dicarboxylic acid, isodecyl isobutyl ester of cyclohexane-1,4-dicarboxylic acid, n-undecyl isobutyl ester of cyclohexane-1,4-dicarboxylic acid, isoundecyl isobutyl ester of cyclohexane-1,4-dicarboxylic acid, n-dodecyl isobutyl ester of cyclohexane-1,4-dicarboxylic acid, isododecyl isobutyl ester of cyclohexane-1,4-dicarboxylic acid, n-tridecyl isobutyl ester of cyclohexane-1,4-dicarboxylic acid, isotridecyl isobutyl ester of cyclohexane-1,4-dicarboxylic acid, n-hexyl glycol ester of cyclohexane-1,4-dicarboxylic acid, isohexyl glycol ester of cyclohexane-1,4-dicarboxylic acid, n-heptyl glycol ester of cyclohexane-1,4-dicarboxylic acid, isoheptyl glycol ester of cyclohexane-1,4-dicarboxylic acid, n-octyl glycol ester of cyclohexane-1,4-dicarboxylic acid, isooctyl glycol ester of cyclohexane-1,4-dicarboxylic acid, 2-ethylhexyl glycol ester of cyclohexane-1,4-dicarboxylic acid, n-nonyl glycol ester of cyclohexane-1,4-dicarboxylic acid, isononyl glycol ester of cyclohexane-1,4-dicarboxylic acid, 2-propylheptyl glycol ester of cyclohexane-1,4-dicarboxylic acid, n-decyl glycol ester of cyclohexane-1,4-dicarboxylic acid, isodecyl glycol ester of cyclohexane-1,4-dicarboxylic acid, n-undecyl glycol ester of cyclohexane-1,4-dicarboxylic acid, isoundecyl glycol ester of cyclohexane-1,4-dicarboxylic acid, n-dodecyl glycol ester of cyclohexane-1,4-dicarboxylic acid, isododecyl glycol ester of cyclohexane-1,4-dicarboxylic acid, n-tridecyl glycol ester of cyclohexane-1,4-dicarboxylic acid, isotridecyl glycol ester of cyclohexane-1,4-dicarboxylic acid, isohexyl n-hexyl ester of cyclohexane-1,4-dicarboxylic acid, n-heptyl n-hexyl ester of cyclohexane-1,4-dicarboxylic acid, isoheptyl n-hexyl ester of cyclohexane-1,4-dicarboxylic acid, n-octyl n-hexyl ester of cyclohexane-1,4-dicarboxylic acid, isooctyl n-hexyl ester of cyclohexane-1,4-dicarboxylic acid, 2-ethylhexyl n-hexyl ester of cyclohexane-1,4-dicarboxylic acid, n-nonyl n-hexyl ester of cyclohexane-1,4-dicarboxylic acid, isononyl n-hexyl ester of cyclohexane-1,4-dicarboxylic acid, 2-propylheptyl n-hexyl ester of cyclohexane-1,4-dicarboxylic acid, n-decyl n-hexyl ester of cyclohexane-1,4-dicarboxylic acid, isodecyl n-hexyl ester of cyclohexane-1,4-dicarboxylic acid, n-undecyl n-hexyl ester of cyclohexane-1,4-dicarboxylic acid, isoundecyl n-hexyl ester of cyclohexane-1,4-dicarboxylic acid, n-dodecyl n-hexyl ester of cyclohexane-1,4-dicarboxylic acid, isododecyl n-hexyl ester of cyclohexane-1,4-dicarboxylic acid, n-tridecyl n-hexyl ester of cyclohexane-1,4-dicarboxylic acid, isotridecyl n-hexyl ester of cyclohexane-1,4-dicarboxylic acid, n-heptyl isohexyl ester of cyclohexane-1,4-dicarboxylic acid, isoheptyl isohexyl ester of cyclohexane-1,4-dicarboxylic acid, n-octyl isohexyl ester of cyclohexane-1,4-dicarboxylic acid, isooctyl isohexyl ester of cyclohexane-1,4-dicarboxylic acid, 2-ethylhexyl isohexyl ester of cyclohexane-1,4-dicarboxylic acid, n-nonyl isohexyl ester of cyclohexane-1,4-dicarboxylic acid, isononyl isohexyl ester of cyclohexane-1,4-dicarboxylic acid, 2-propylheptyl isohexyl ester of cyclohexane-1,4-dicarboxylic acid, n-decyl isohexyl ester of cyclohexane-1,4-dicarboxylic acid, isodecyl isohexyl ester of cyclohexane-1,4-dicarboxylic acid, n-undecyl isohexyl ester of cyclohexane-1,4-dicarboxylic acid, isoundecyl isohexyl ester of cyclohexane-1,4-dicarboxylic acid, n-dodecyl isohexyl ester of cyclohexane-1,4-dicarboxylic acid, isododecyl isohexyl ester of cyclohexane-1,4-dicarboxylic acid, n-tridecyl isohexyl ester of cyclohexane-1,4-dicarboxylic acid, isotridecyl isohexyl ester of cyclohexane-1,4-dicarboxylic acid, isoheptyl n-heptyl ester of cyclohexane-1,4-dicarboxylic acid, n-octyl n-heptyl ester of cyclohexane-1,4-dicarboxylic acid, isooctyl n-heptyl ester of cyclohexane-1,4-dicarboxylic acid, 2-ethylhexyl n-heptyl ester of cyclohexane-1,4-dicarboxylic acid, n-nonyl n-heptyl ester of cyclohexane-1,4-dicarboxylic acid, isononyl n-heptyl ester of cyclohexane-1,4-dicarboxylic acid, 2-propylheptyl n-heptyl ester of cyclohexane-1,4-dicarboxylic acid, n-decyl n-heptyl ester of cyclohexane-1,4-dicarboxylic acid, isodecyl n-heptyl ester of cyclohexane-1,4-dicarboxylic acid, n-undecyl n-heptyl ester of cyclohexane-1,4-dicarboxylic acid, isoundecyl n-heptyl ester of cyclohexane-1,4-dicarboxylic acid, n-dodecyl n-heptyl ester of cyclohexane-1,4-dicarboxylic acid, isododecyl n-heptyl ester of cyclohexane-1,4-dicarboxylic acid, n-tridecyl n-heptyl ester of cyclohexane-1,4-dicarboxylic acid, isotridecyl n-heptyl ester of cyclohexane-1,4-dicarboxylic acid, n-octyl isoheptyl ester of cyclohexane-1,4-dicarboxylic acid, isooctyl isoheptyl ester of cyclohexane-1,4-dicarboxylic acid, 2-ethylhexyl isoheptyl ester of cyclohexane-1,4-dicarboxylic acid, n-nonyl isoheptyl ester of cyclohexane-1,4-dicarboxylic acid, isononyl isoheptyl ester of cyclohexane-1,4-dicarboxylic acid, 2-propylheptyl isoheptyl ester of cyclohexane-1,4-dicarboxylic acid, n-decyl isoheptyl ester of cyclohexane-1,4-dicarboxylic acid, isodecyl isoheptyl ester of cyclohexane-1,4-dicarboxylic acid, n-undecyl isoheptyl ester of cyclohexane-1,4-dicarboxylic acid, isoundecyl isoheptyl ester of cyclohexane-1,4-dicarboxylic acid, n-dodecyl isoheptyl ester of cyclohexane-1,4-dicarboxylic acid, isododecyl isoheptyl ester of cyclohexane-1,4-dicarboxylic acid, n-tridecyl isoheptyl ester of cyclohexane-1,4-dicarboxylic acid, isotridecyl isoheptyl ester of cyclohexane-1,4-dicarboxylic acid, isooctyl n-octyl ester of cyclohexane-1,4-dicarboxylic acid, 2-ethylhexyl n-octyl ester of cyclohexane-1,4-dicarboxylic acid, n-nonyl n-octyl ester of cyclohexane-1,4-dicarboxylic acid, isononyl n-octyl ester of cyclohexane-1,4-dicarboxylic acid, 2-propylheptyl n-octyl ester of cyclohexane-1,4-dicarboxylic acid, n-decyl n-octyl ester of cyclohexane-1,4-dicarboxylic acid, isodecyl n-octyl ester of cyclohexane-1,4-dicarboxylic acid, n-undecyl n-octyl ester of cyclohexane-1,4-dicarboxylic acid, isoundecyl n-octyl ester of cyclohexane-1,4-dicarboxylic acid, n-dodecyl n-octyl ester of cyclohexane-1,4-dicarboxylic acid, isododecyl n-octyl ester of cyclohexane-1,4-dicarboxylic acid, n-tridecyl n-octyl ester of cyclohexane-1,4-dicarboxylic acid, isotridecyl n-octyl ester of cyclohexane-1,4-dicarboxylic acid, 2-ethylhexyl isooctyl ester of cyclohexane-1,4-dicarboxylic acid, n-nonyl isooctyl ester of cyclohexane-1,4-dicarboxylic acid, isononyl isooctyl ester of cyclohexane-1,4-dicarboxylic acid, 2-propylheptyl isooctyl ester of cyclohexane-1,4-dicarboxylic acid, n-decyl isooctyl ester of cyclohexane-1,4-dicarboxylic acid, isodecyl isooctyl ester of cyclohexane-1,4-dicarboxylic acid, n-undecyl isooctyl ester of cyclohexane-1,4-dicarboxylic acid, isoundecyl isooctyl ester of cyclohexane-1,4-dicarboxylic acid, n-dodecyl isooctyl ester of cyclohexane-1,4-dicarboxylic acid, isododecyl isooctyl ester of cyclohexane-1,4-dicarboxylic acid, n-tridecyl isooctyl ester of cyclohexane-1,4-dicarboxylic acid, isotridecyl isooctyl ester of cyclohexane-1,4-dicarboxylic acid, n-nonyl 2-ethylhexyl ester of cyclohexane-1,4-dicarboxylic acid, isononyl 2-ethylhexyl ester of cyclohexane-1,4-dicarboxylic acid, 2-propylheptyl 2-ethylhexyl ester of cyclohexane-1,4-dicarboxylic acid, n-decyl 2-ethylhexyl ester of cyclohexane-1,4-dicarboxylic acid, isodecyl 2-ethylhexyl ester of cyclohexane-1,4-dicarboxylic acid, n-undecyl 2-ethylhexyl ester of cyclohexane-1,4-dicarboxylic acid, isoundecyl 2-ethylhexyl ester of cyclohexane-1,4-dicarboxylic acid, n-dodecyl 2-ethylhexyl ester of cyclohexane-1,4-dicarboxylic acid, isododecyl 2-ethylhexyl ester of cyclohexane-1,4-dicarboxylic acid, n-tridecyl 2-ethylhexyl ester of cyclohexane-1,4-dicarboxylic acid, isotridecyl 2-ethylhexyl ester of cyclohexane-1,4-dicarboxylic acid, isononyl n-nonyl ester of cyclohexane-1,4-dicarboxylic acid, 2-propylheptyl n-nonyl ester of cyclohexane-1,4-dicarboxylic acid, n-decyl n-nonyl ester of cyclohexane-1,4-dicarboxylic acid, isodecyl n-nonyl ester of cyclohexane-1,4-dicarboxylic acid, n-undecyl n-nonyl ester of cyclohexane-1,4-dicarboxylic acid, isoundecyl n-nonyl ester of cyclohexane-1,4-dicarboxylic acid, n-dodecyl n-nonyl ester of cyclohexane-1,4-dicarboxylic acid, isododecyl n-nonyl ester of cyclohexane-1,4-dicarboxylic acid, n-tridecyl n-nonyl ester of cyclohexane-1,4-dicarboxylic acid, isotridecyl n-nonyl ester of cyclohexane-1,4-dicarboxylic acid, 2-propylheptyl isononyl ester of cyclohexane-1,4-dicarboxylic acid, n-decyl isononyl ester of cyclohexane-1,4-dicarboxylic acid, isodecyl isononyl ester of cyclohexane-1,4-dicarboxylic acid, n-undecyl isononyl ester of cyclohexane-1,4-dicarboxylic acid, isoundecyl isononyl ester of cyclohexane-1,4-dicarboxylic acid, n-dodecyl isononyl ester of cyclohexane-1,4-dicarboxylic acid, isododecyl isononyl ester of cyclohexane-1,4-dicarboxylic acid, n-tridecyl isononyl ester of cyclohexane-1,4-dicarboxylic acid, isotridecyl isononyl ester of cyclohexane-1,4-dicarboxylic acid, n-decyl 2-propylheptyl ester of cyclohexane-1,4-dicarboxylic acid, isodecyl 2-propylheptyl ester of cyclohexane-1,4-dicarboxylic acid, n-undecyl 2-propylheptyl ester of cyclohexane-1,4-dicarboxylic acid, isoundecyl 2-propylheptyl ester of cyclohexane-1,4-dicarboxylic acid, n-dodecyl 2-propylheptyl ester of cyclohexane-1,4-dicarboxylic acid, isododecyl 2-propylheptyl ester of cyclohexane-1,4-dicarboxylic acid, n-tridecyl 2-propylheptyl ester of cyclohexane-1,4-dicarboxylic acid, isotridecyl 2-propylheptyl ester of cyclohexane-1,4-dicarboxylic acid, isodecyl n-decyl ester of cyclohexane-1,4-dicarboxylic acid, n-undecyl n-decyl ester of cyclohexane-1,4-dicarboxylic acid, isoundecyl n-decyl ester of cyclohexane-1,4-dicarboxylic acid, n-dodecyl n-decyl ester of cyclohexane-1,4-dicarboxylic acid, isododecyl n-decyl ester of cyclohexane-1,4-dicarboxylic acid, n-tridecyl n-decyl ester of cyclohexane-1,4-dicarboxylic acid, isotridecyl n-decyl ester of cyclohexane-1,4-dicarboxylic acid, n-undecyl isodecyl ester of cyclohexane-1,4-dicarboxylic acid, isoundecyl isodecyl ester of cyclohexane-1,4-dicarboxylic acid, n-dodecyl isodecyl ester of cyclohexane-1,4-dicarboxylic acid, isododecyl isodecyl ester of cyclohexane-1,4-dicarboxylic acid, n-tridecyl isodecyl ester of cyclohexane-1,4-dicarboxylic acid, isotridecyl isodecyl ester of cyclohexane-1,4-dicarboxylic acid, isoundecyl n-undecyl ester of cyclohexane-1,4-dicarboxylic acid, n-dodecyl n-undecyl ester of cyclohexane-1,4-dicarboxylic acid, isododecyl n-undecyl ester of cyclohexane-1,4-dicarboxylic acid, n-tridecyl n-undecyl ester of cyclohexane-1,4-dicarboxylic acid, isotridecyl n-undecyl ester of cyclohexane-1,4-dicarboxylic acid, n-dodecyl isoundecyl ester of cyclohexane-1,4-dicarboxylic acid, isododecyl isoundecyl ester of cyclohexane-1,4-dicarboxylic acid, n-tridecyl isoundecyl ester of cyclohexane-1,4-dicarboxylic acid, isotridecyl isoundecyl ester of cyclohexane-1,4-dicarboxylic acid, isododecyl n-dodecyl ester of cyclohexane-1,4-dicarboxylic acid, n-tridecyl n-dodecyl ester of cyclohexane-1,4-dicarboxylic acid, isotridecyl n-dodecyl ester of cyclohexane-1,4-dicarboxylic acid, n-tridecyl isododecyl ester of cyclohexane-1,4-dicarboxylic acid, isotridecyl isododecyl ester of cyclohexane-1,4-dicarboxylic acid, isotridecyl n-tridecyl ester of cyclohexane-1,4-dicarboxylic acid, methyl cyclohexyl ester of cyclohexane-1,4-dicarboxylic acid, ethyl cyclohexyl ester of cyclohexane-1,4-dicarboxylic acid, n-propyl cyclohexyl ester of cyclohexane-1,4-dicarboxylic acid, isopropyl cyclohexyl ester of cyclohexane-1,4-dicarboxylic acid, n-butyl cyclohexyl ester of cyclohexane-1,4-dicarboxylic acid, tert-butyl cyclohexyl ester of cyclohexane-1,4-dicarboxylic acid, isobutyl cyclohexyl ester of cyclohexane-1,4-dicarboxylic acid, glycol cyclohexyl ester of cyclohexane-1,4-dicarboxylic acid, n-hexyl cyclohexyl ester of cyclohexane-1,4-dicarboxylic acid, isohexyl cyclohexyl ester of cyclohexane-1,4-dicarboxylic acid, n-heptyl cyclohexyl ester of cyclohexane-1,4-dicarboxylic acid, isoheptyl cyclohexyl ester of cyclohexane-1,4-dicarboxylic acid, n-octyl cyclohexyl ester of cyclohexane-1,4-dicarboxylic acid, isooctyl cyclohexyl ester of cyclohexane-1,4-dicarboxylic acid, 2-ethylhexyl cyclohexyl ester of cyclohexane-1,4-dicarboxylic acid, n-nonyl cyclohexyl ester of cyclohexane-1,4-dicarboxylic acid, isononyl cyclohexyl ester of cyclohexane-1,4-dicarboxylic acid, 2-propylheptyl cyclohexyl ester of cyclohexane-1,4-dicarboxylic acid, n-decyl cyclohexyl ester of cyclohexane-1,4-dicarboxylic acid, isodecyl cyclohexyl ester of cyclohexane-1,4-dicarboxylic acid, n-undecyl cyclohexyl ester of cyclohexane-1,4-dicarboxylic acid, isoundecyl cyclohexyl ester of cyclohexane-1,4-dicarboxylic acid, n-dodecyl cyclohexyl ester of cyclohexane-1,4-dicarboxylic acid, isododecyl cyclohexyl ester of cyclohexane-1,4-dicarboxylic acid, n-tridecyl cyclohexyl ester of cyclohexane-1,4-dicarboxylic acid, isotridecyl cyclohexyl ester of cyclohexane-1,4-dicarboxylic acid, methyl isopentyl ester of cyclohexane-1,4-dicarboxylic acid, ethyl isopentyl ester of cyclohexane-1,4-dicarboxylic acid, n-propyl isopentyl ester of cyclohexane-1,4-dicarboxylic acid, isopropyl isopentyl ester of cyclohexane-1,4-dicarboxylic acid, n-butyl isopentyl ester of cyclohexane-1,4-dicarboxylic acid, tert-butyl isopentyl ester of cyclohexane-1,4-dicarboxylic acid, isobutyl isopentyl ester of cyclohexane-1,4-dicarboxylic acid, glycol isopentyl ester of cyclohexane-1,4-dicarboxylic acid, n-hexyl isopentyl ester of cyclohexane-1,4-dicarboxylic acid, isohexyl isopentyl ester of cyclohexane-1,4-dicarboxylic acid, n-heptyl isopentyl ester of cyclohexane-1,4-dicarboxylic acid, isoheptyl isopentyl ester of cyclohexane-1,4-dicarboxylic acid, n-octyl isopentyl ester of cyclohexane-1,4-dicarboxylic acid, isooctyl isopentyl ester of cyclohexane-1,4-dicarboxylic acid, 2-ethylhexyl isopentyl ester of cyclohexane-1,4-dicarboxylic acid, n-nonyl isopentyl ester of cyclohexane-1,4-dicarboxylic acid, isononyl isopentyl ester of cyclohexane-1,4-dicarboxylic acid, 2-propylheptyl isopentyl ester of cyclohexane-1,4-dicarboxylic acid, n-decyl isopentyl ester of cyclohexane-1,4-dicarboxylic acid, isodecyl isopentyl ester of cyclohexane-1,4-dicarboxylic acid, n-undecyl isopentyl ester of cyclohexane-1,4-dicarboxylic acid, isoundecyl isopentyl ester of cyclohexane-1,4-dicarboxylic acid, n-dodecyl isopentyl ester of cyclohexane-1,4-dicarboxylic acid, isododecyl isopentyl ester of cyclohexane-1,4-dicarboxylic acid, n-tridecyl isopentyl ester of cyclohexane-1,4-dicarboxylic acid, isotridecyl isopentyl ester of cyclohexane-1,4-dicarboxylic acid, methyl n-pentyl ester of cyclohexane-1,4-dicarboxylic acid, ethyl n-pentyl ester of cyclohexane-1,4-dicarboxylic acid, n-propyl n-pentyl ester of cyclohexane-1,4-dicarboxylic acid, isopropyl n-pentyl ester of cyclohexane-1,4-dicarboxylic acid, n-butyl n-pentyl ester of cyclohexane-1,4-dicarboxylic acid, tert-butyl n-pentyl ester of cyclohexane-1,4-dicarboxylic acid, isobutyl n-pentyl ester of cyclohexane-1,4-dicarboxylic acid, glycol n-pentyl ester of cyclohexane-1,4-dicarboxylic acid, n-hexyl n-pentyl ester of cyclohexane-1,4-dicarboxylic acid, isohexyl n-pentyl ester of cyclohexane-1,4-dicarboxylic acid, n-heptyl n-pentyl ester of cyclohexane-1,4-dicarboxylic acid, isoheptyl n-pentyl ester of cyclohexane-1,4-dicarboxylic acid, n-octyl n-pentyl ester of cyclohexane-1,4-dicarboxylic acid, isooctyl n-pentyl ester of cyclohexane-1,4-dicarboxylic acid, 2-ethylhexyl n-pentyl ester of cyclohexane-1,4-dicarboxylic acid, n-nonyl n-pentyl ester of cyclohexane-1,4-dicarboxylic acid, isononyl n-pentyl ester of cyclohexane-1,4-dicarboxylic acid, 2-propylheptyl n-pentyl ester of cyclohexane-1,4-dicarboxylic acid, n-decyl n-pentyl ester of cyclohexane-1,4-dicarboxylic acid, isodecyl n-pentyl ester of cyclohexane-1,4-dicarboxylic acid, n-undecyl n-pentyl ester of cyclohexane-1,4-dicarboxylic acid, isoundecyl n-pentyl ester of cyclohexane-1,4-dicarboxylic acid, n-dodecyl n-pentyl ester of cyclohexane-1,4-dicarboxylic acid, isododecyl n-pentyl ester of cyclohexane-1,4-dicarboxylic acid, n-tridecyl n-pentyl ester of cyclohexane-1,4-dicarboxylic acid, isotridecyl n-pentyl ester of cyclohexane-1,4-dicarboxylic acid;

alkyl esters of cyclohexane-1,3-dicarboxylic acid, such as, for example, monomethyl ester of cyclohexane-1,3-dicarboxylic acid, dimethyl ester of cyclohexane-1,3-dicarboxylic acid, diethyl ester of cyclohexane-1,3-dicarboxylic acid, di-n-propyl ester of cyclohexane-1,3-dicarboxylic acid, di-n-butyl ester of cyclohexane-1,3-dicarboxylic acid, di-tert-butyl ester of cyclohexane-1,3-dicarboxylic acid, diisobutyl ester of cyclohexane-1,3-dicarboxylic acid, monoglycol ester of cyclohexane-1,3-dicarboxylic acid, diglycol ester of cyclohexane-1,3-dicarboxylic acid, di-n-octyl ester of cyclohexane-1,3-dicarboxylic acid, diisooctyl ester of cyclohexane-1,3-dicarboxylic acid, di-2-ethylhexyl ester of cyclohexane-1,3-dicarboxylic acid, di-n-nonyl ester of cyclohexane-1,3-dicarboxylic acid, diisononyl ester of cyclohexane-1,3-dicarboxylic acid, di-n-decyl ester of cyclohexane-1,3-dicarboxylic acid, diisodecyl ester of cyclohexane-1,3-dicarboxylic acid, di-n-undecyl ester of cyclohexane-1,3-dicarboxylic acid, diisododecyl ester of cyclohexane-1,3-dicarboxylic acid, di-n-octadecyl ester of cyclohexane-1,3-dicarboxylic acid, diisooctadecyl ester of cyclohexane-1,3-dicarboxylic acid, di-n-eicosyl ester of cyclohexane-1,3-dicarboxylic acid, monocyclohexyl ester of cyclohexane-1,3-dicarboxylic acid, dicyclohexyl ester of cyclohexane-1,3-dicarboxylic acid;

diisopropyl ester of cyclohexane-1,3-dicarboxylic acid, di-n-hexyl ester of cyclohexane-1,3-dicarboxylic acid, diisohexyl ester of cyclohexane-1,3-dicarboxylic acid, di-n-heptyl ester of cyclohexane-1,3-dicarboxylic acid, diisoheptyl ester of cyclohexane-1,3-dicarboxylic acid, di-2-propylheptyl ester of cyclohexane-1,3-dicarboxylic acid, diisoundecyl ester of cyclohexane-1,3-dicarboxylic acid, di-n-dodecyl ester of cyclohexane-1,3-dicarboxylic acid, di-n-tridecyl ester of cyclohexane-1,3-dicarboxylic acid, diisotridecyl ester of cyclohexane-1,3-dicarboxylic acid, di-n-pentyl ester of cyclohexane-1,3-dicarboxylic acid, diisopentyl ester of cyclohexane-1,3-dicarboxylic acid;

alkyl esters of cyclohexane-1,2,4-tricarboxylic acid, such as, for example, monomethyl ester of cyclohexane-1,2,4-tricarboxylic acid, dimethyl ester of cyclohexane-1,2,4-tricarboxylic acid, diethyl ester of cyclohexane-1,2,4-tricarboxylic acid, di-n-propyl ester of cyclohexane-1,2,4-tricarboxylic acid, diisopropyl ester of cyclohexane-1,2,4-tricarboxylic acid, di-n-butyl ester of cyclohexane-1,2,4-tricarboxylic acid, di-tert-butyl ester of cyclohexane-1,2,4-tricarboxylic acid, diisobutyl ester of cyclohexane-1,2,4-tricarboxylic acid, monoglycol ester of cyclohexane-1,2,4-tricarboxylic acid, diglycol ester of cyclohexane-1,2,4-tricarboxylic acid, di-n-octyl ester of cyclohexane-1,2,4-tricarboxylic acid, diisooctyl ester of cyclohexane-1,2,4-tricarboxylic acid, di-2-ethylhexyl ester of cyclohexane-1,2,4-tricarboxylic acid, di-n-nonyl ester of cyclohexane-1,2,4-tricarboxylic acid, diisononyl ester of cyclohexane-1,2,4-tricarboxylic acid, di-n-decyl ester of cyclohexane-1,2,4-tricarboxylic acid, diisodecyl ester of cyclohexane-1,2,4-tricarboxylic acid, di-n-undecyl ester of cyclohexane-1,2,4-tricarboxylic acid, diisododecyl ester of cyclohexane-1,2,4-tricarboxylic acid, di-n-octadecyl ester of cyclohexane-1,2,4-tricarboxylic acid, diisooctadecyl ester of cyclohexane-1,2,4-tricarboxylic acid, di-n-eicosyl ester of cyclohexane-1,2,4-tricarboxylic acid, monocyclohexyl ester of cyclohexane-1,2,4-tricarboxylic acid, dicyclohexyl ester of cyclohexane-1,2,4-tricarboxylic acid and trimethyl ester of cyclohexane-1,2,4-tricarboxylic acid, triethyl ester of cyclohexane-1,2,4-tricarboxylic acid, tri-n-propyl ester of cyclohexane-1,2,4-tricarboxylic acid, tri-n-butyl ester of cyclohexane-1,2,4-tricarboxylic acid, tri-tert-butyl ester of cyclohexane-1,2,4-tricarboxylic acid, triisobutyl ester of cyclohexane-1,2,4-tricarboxylic acid, triglycol ester of cyclohexane-1,2,4-tricarboxylic acid, tri-n-octyl ester of cyclohexane-1,2,4-tricarboxylic acid, triisooctyl ester of cyclohexane-1,2,4-tricarboxylic acid, tri-2-ethylhexyl ester of cyclohexane-1,2,4-tricarboxylic acid, tri-n-nonyl ester of cyclohexane-1,2,4-tricarboxylic acid, triisononyl ester of cyclohexane-1,2,4-tricarboxylic acid, tri-n-decyl ester of cyclohexane-1,2,4-tricarboxylic acid, triisododecyl ester of cyclohexane-1,2,4-tricarboxylic acid, tri-n-undecyl ester of cyclohexane-1,2,4-tricarboxylic acid, triisododecyl ester of cyclohexane-1,2,4-tricarboxylic acid, tri-n-octadecyl ester of cyclohexane-1,2,4-tricarboxylic acid, triisooctadecyl ester of cyclohexane-1,2,4-tricarboxylic acid, tri-n-eicosyl ester of cyclohexane-1,2,4-tricarboxylic acid, tricyclohexyl ester of cyclohexane-1,2,4-tricarboxylic acid, di-n-hexyl ester of cyclohexane-1,2,4-tricarboxylic acid, diisohexyl ester of cyclohexane-1,2,4-tricarboxylic acid, tri-n-hexyl ester of cyclohexane-1,2,4-tricarboxylic acid, triisohexyl ester of cyclohexane-1,2,4-tricarboxylic acid, di-n-heptyl ester of cyclohexane-1,2,4-tricarboxylic acid, diisoheptyl ester of cyclohexane-1,2,4-tricarboxylic acid, tri-n-heptyl ester of cyclohexane-1,2,4-tricarboxylic acid, triisoheptyl ester of cyclohexane-1,2,4-tricarboxylic acid, di-n-pentyl ester of cyclohexane-1,2,4-tricarboxylic acid, diisopentyl ester of cyclohexane-1,2,4-tricarboxylic acid, tri-n-pentyl ester of cyclohexane-1,2,4-tricarboxylic acid, triisopentyl ester of cyclohexane-1,2,4-tricarboxylic acid, di-n-tridecyl ester of cyclohexane-1,2,4-tricarboxylic acid, diisotridecyl ester of cyclohexane-1,2,4-tricarboxylic acid, tri-n-tridecyl ester of cyclohexane-1,2,4-tricarboxylic acid, triisotridecyl ester of cyclohexane-1,2,4-tricarboxylic acid, di-n-dodecyl ester of cyclohexane-1,2,4-tricarboxylic acid, diisoundecyl ester of cyclohexane-1,2,4-tricarboxylic acid, tri-n-dodecyl ester of cyclohexane-1,2,4-tricarboxylic acid, triisoundecyl ester of cyclohexane-1,2,4-tricarboxylic acid, triisopropyl ester of cyclohexane-1,2,4-tricarboxylic acid;

alkyl esters of cyclohexane-1,3,5-tricarboxylic acid, such as, for example, monomethyl ester of cyclohexane-1,3,5-tricarboxylic acid, dimethyl ester of cyclohexane-1,3,5-tricarboxylic acid, diethyl ester of cyclohexane-1,3,5-tricarboxylic acid, di-n-propyl ester of cyclohexane-1,3,5-tricarboxylic acid, di-n-butyl ester of cyclohexane-1,3,5-tricarboxylic acid, di-tert-butyl ester of cyclohexane-1,3,5-tricarboxylic acid, diisobutyl ester of cyclohexane-1,3,5-tricarboxylic acid, monoglycol ester of cyclohexane-1,3,5-tricarboxylic acid, diglycol ester of cyclohexane-1,3,5-tricarboxylic acid, di-n-octyl ester of cyclohexane-1,3,5-tricarboxylic acid, diisooctyl ester of cyclohexane-1,3,5-tricarboxylic acid, di-2-ethylhexyl ester of cyclohexane-1,3,5-tricarboxylic acid, di-n-nonyl ester of cyclohexane-1,3,5-tricarboxylic acid, diisononyl ester of cyclohexane-1,3,5-tricarboxylic acid, di-n-decyl ester of cyclohexane-1,3,5-tricarboxylic acid, diisodecyl ester of cyclohexane-1,3,5-tricarboxylic acid, di-n-undecyl ester of cyclohexane-1,3,5-tricarboxylic acid, diisododecyl ester of cyclohexane-1,3,5-tricarboxylic acid, di-n-octadecyl ester of cyclohexane-1,3,5-tricarboxylic acid, diisooctadecyl ester of cyclohexane-1,3,5-tricarboxylic acid, di-n-eicosyl ester of cyclohexane-1,3,5-tricarboxylic acid, monocyclohexyl ester of cyclohexane-1,3,5-tricarboxylic acid, dicyclohexyl ester of cyclohexane-1,3,5-tricarboxylic acid, trimethyl ester of cyclohexane-1,3,5-tricarboxylic acid, triethyl ester of cyclohexane-1,3,5-tricarboxylic acid, tri-n-propyl ester of cyclohexane-1,3,5-tricarboxylic acid, tri-n-butyl ester of cyclohexane-1,3,5-tricarboxylic acid, tri-tert-butyl ester of cyclohexane-1,3,5-tricarboxylic acid, triisobutyl ester of cyclohexane-1,3,5-tricarboxylic acid, triglycol ester of cyclohexane-1,3,5-tricarboxylic acid, tri-n-octyl ester of cyclohexane-1,3,5-tricarboxylic acid, triisooctyl ester of cyclohexane-1,3,5-tricarboxylic acid, tri-2-ethylhexyl ester of cyclohexane-1,3,5-tricarboxylic acid, tri-n-nonyl ester of cyclohexane-1,3,5-tricarboxylic acid, triisononyl ester of cyclohexane-1,3,5-tricarboxylic acid, tri-n-decyl ester of cyclohexane-1,3,5-tricarboxylic acid, triisododecyl ester of cyclohexane-1,3,5-tricarboxylic acid, tri-n-undecyl ester of cyclohexane-1,3,5-tricarboxylic acid, triisododecyl ester of cyclohexane-1,3,5-tricarboxylic acid, tri-n-octadecyl ester of cyclohexane-1,3,5-tricarboxylic acid, triisooctadecyl ester of cyclohexane-1,3,5-tricarboxylic acid, tri-n-eicosyl ester of cyclohexane-1,3,5-tricarboxylic acid, tricyclohexyl ester of cyclohexane-1,3,5-tricarboxylic acid, di-n-hexyl ester of cyclohexane-1,3,5-tricarboxylic acid, diisohexyl ester of cyclohexane-1,3,5-tricarboxylic acid, tri-n-hexyl ester of cyclohexane-1,3,5-tricarboxylic acid, triisohexyl ester of cyclohexane-1,3,5-tricarboxylic acid, di-n-heptyl ester of cyclohexane-1,3,5-tricarboxylic acid, diisoheptyl ester of cyclohexane-1,3,5-tricarboxylic acid, tri-n-heptyl ester of cyclohexane-1,3,5-tricarboxylic acid, triisoheptyl ester of cyclohexane-1,3,5-tricarboxylic acid, di-n-pentyl ester of cyclohexane-1,3,5-tricarboxylic acid, diisopentyl ester of cyclohexane-1,3,5-tricarboxylic acid, tri-n-pentyl ester of cyclohexane-1,3,5-tricarboxylic acid, triisopentyl ester of cyclohexane-1,3,5-tricarboxylic acid, di-n-tridecyl ester of cyclohexane-1,3,5-tricarboxylic acid, diisotridecyl ester of cyclohexane-1,3,5-tricarboxylic acid, tri-n-tridecyl ester of cyclohexane-1,3,5-tricarboxylic acid, triisotridecyl ester of cyclohexane-1,3,5-tricarboxylic acid, di-n-dodecyl ester of cyclohexane-1,3,5-tricarboxylic acid, diisoundecyl ester of cyclohexane-1,3,5-tricarboxylic acid, tri-n-dodecyl ester of cyclohexane-1,3,5-tricarboxylic acid, triisoundecyl ester of cyclohexane-1,3,5-tricarboxylic acid, triisopropyl ester of cyclohexane-1,3,5-tricarboxylic acid;

alkyl esters of cyclohexane-1,2,3-tricarboxylic acid, such as, for example, monomethyl ester of cyclohexane-1,2,3-tricarboxylic acid, dimethyl ester of cyclohexane-1,2,3-tricarboxylic acid, diethyl ester of cyclohexane-1,2,3-tricarboxylic acid, di-n-propyl ester of cyclohexane-1,2,3-tricarboxylic acid, di-n-butyl ester of cyclohexane-1,2,3-tricarboxylic acid, di-tert-butyl ester of cyclohexane-1,2,3-tricarboxylic acid, diisobutyl ester of cyclohexane-1,2,3-tricarboxylic acid, monoglycol ester of cyclohexane-1,2,3-tricarboxylic acid, diglycol ester of cyclohexane-1,2,3-tricarboxylic acid, di-n-octyl ester of cyclohexane-1,2,3-tricarboxylic acid, diisooctyl ester of cyclohexane-1,2,3-tricarboxylic acid, di-2-ethylhexyl ester of cyclohexane-1,2,3-tricarboxylic acid, di-n-nonyl ester of cyclohexane-1,2,3-tricarboxylic acid, diisononyl ester of cyclohexane-1,2,3-tricarboxylic acid, di-n-decyl ester of cyclohexane-1,2,3-tricarboxylic acid, diisodecyl ester of cyclohexane-1,2,3-tricarboxylic acid, di-n-undecyl ester of cyclohexane-1,2,3-tricarboxylic acid, diisododecyl ester of cyclohexane-1,2,3-tricarboxylic acid, di-n-octadecyl ester of cyclohexane-1,2,3-tricarboxylic acid, diisooctadecyl ester of cyclohexane-1,2,3-tricarboxylic acid, di-n-eicosyl ester of cyclohexane-1,2,3-tricarboxylic acid, monocyclohexyl ester of cyclohexane-1,2,3-tricarboxylic acid, dicyclohexyl ester of cyclohexane-1,2,3-tricarboxylic acid, trimethyl ester of cyclohexane-1,2,3-tricarboxylic acid, triethyl ester of cyclohexane-1,2,3-tricarboxylic acid, tri-n-propyl ester of cyclohexane-1,2,3-tricarboxylic acid, tri-n-butyl ester of cyclohexane-1,2,3-tricarboxylic acid, tri-tert-butyl ester of cyclohexane-1,2,3-tricarboxylic acid, triisobutyl ester of cyclohexane-1,2,3-tricarboxylic acid, triglycol ester of cyclohexane-1,2,3-tricarboxylic acid, tri-n-octyl ester of cyclohexane-1,2,3-tricarboxylic acid, triisooctyl ester of cyclohexane-1,2,3-tricarboxylic acid, tri-2-ethylhexyl ester of cyclohexane-1,2,3-tricarboxylic acid, tri-n-nonyl ester of cyclohexane-1,2,3-tricarboxylic acid, triisononyl ester of cyclohexane-1,2,3-tricarboxylic acid, tri-n-decyl ester of cyclohexane-1,2,3-tricarboxylic acid, triisododecyl ester of cyclohexane-1,2,3-tricarboxylic acid, tri-n-undecyl ester of cyclohexane-1,2,3-tricarboxylic acid, triisododecyl ester of cyclohexane-1,2,3-tricarboxylic acid, tri-n-octadecyl ester of cyclohexane-1,2,3-tricarboxylic acid, triisooctadecyl ester of cyclohexane-1,2,3-tricarboxylic acid, tri-n-eicosyl ester of cyclohexane-1,2,3-tricarboxylic acid, tricyclohexyl ester of cyclohexane-1,2,3-tricarboxylic acid, di-n-hexyl ester of cyclohexane-1,2,3-tricarboxylic acid, diisohexyl ester of cyclohexane-1,2,3-tricarboxylic acid, tri-n-hexyl ester of cyclohexane-1,2,3-tricarboxylic acid, triisohexyl ester of cyclohexane-1,2,3-tricarboxylic acid, di-n-heptyl ester of cyclohexane-1,2,3-tricarboxylic acid, diisoheptyl ester of cyclohexane-1,2,3-tricarboxylic acid, tri-n-heptyl ester of cyclohexane-1,2,3-tricarboxylic acid, triisoheptyl ester of cyclohexane-1,2,3-tricarboxylic acid, di-n-pentyl ester of cyclohexane-1,2,3-tricarboxylic acid, diisopentyl ester of cyclohexane-1,2,3-tricarboxylic acid, tri-n-pentyl ester of cyclohexane-1,2,3-tricarboxylic acid, triisopentyl ester of cyclohexane-1,2,3-tricarboxylic acid, di-n-tridecyl ester of cyclohexane-1,2,3-tricarboxylic acid, diisotridecyl ester of cyclohexane-1,2,3-tricarboxylic acid, tri-n-tridecyl ester of cyclohexane-1,2,3-tricarboxylic acid, triisotridecyl ester of cyclohexane-1,2,3-tricarboxylic acid, di-n-dodecyl ester of cyclohexane-1,2,3-tricarboxylic acid, diisoundecyl ester of cyclohexane-1,2,3-tricarboxylic acid, tri-n-dodecyl ester of cyclohexane-1,2,3-tricarboxylic acid, triisoundecyl ester of cyclohexane-1,2,3-tricarboxylic acid, triisopropyl ester of cyclohexane-1,2,3-tricarboxylic acid;

alkyl esters of cyclohexane-1,2,4,5-tetracarboxylic acid, such as, for example, monomethyl ester of cyclohexane-1,2,4,5-tetracarboxylic acid, dimethyl ester of cyclohexane-1,2,4,5-tetracarboxylic acid, diethyl ester of cyclohexane-1,2,4,5-tetracarboxylic acid, di-n-propyl ester of cyclohexane-1,2,4,5-tetracarboxylic acid, di-n-butyl ester of cyclohexane-1,2,4,5-tetracarboxylic acid, di-tert-butyl ester of cyclohexane-1,2,4,5-tetracarboxylic acid, diisobutyl ester of cyclohexane-1,2,4,5-tetracarboxylic acid, monoglycol ester of cyclohexane-1,2,4,5-tetracarboxylic acid, diglycol ester of cyclohexane-1,2,4,5-tetracarboxylic acid, di-n-octyl ester of cyclohexane-1,2,4,5-tetracarboxylic acid, diisooctyl ester of cyclohexane-1,2,4,5-tetracarboxylic acid, di-2-ethylhexyl ester of cyclohexane-1,2,4,5-tetracarboxylic acid, di-n-nonyl ester of cyclohexane-1,2,4,5-tetracarboxylic acid, diisononyl ester of cyclohexane-1,2,4,5-tetracarboxylic acid, di-n-decyl ester of cyclohexane-1,2,4,5-tetracarboxylic acid, diisodecyl ester of cyclohexane-1,2,4,5-tetracarboxylic acid, di-n-undecyl ester of cyclohexane-1,2,4,5-tetracarboxylic acid, diisododecyl ester of cyclohexane-1,2,4,5-tetracarboxylic acid, di-n-octadecyl ester of cyclohexane-1,2,4,5-tetracarboxylic acid, diisooctadecyl ester of cyclohexane-1,2,4,5-tetracarboxylic acid, di-n-eicosyl ester of cyclohexane-1,2,4,5-tetracarboxylic acid, monocyclohexyl ester of cyclohexane-1,2,4,5-tetracarboxylic acid, trimethyl ester of cyclohexane-1,2,4,5-tetracarboxylic acid, triethyl ester of cyclohexane-1,2,4,5-tetracarboxylic acid, tri-n-propyl ester of cyclohexane-1,2,4,5-tetracarboxylic acid, tri-n-butyl ester of cyclohexane-1,2,4,5-tetracarboxylic acid, tri-tert-butyl ester of cyclohexane-1,2,4,5-tetracarboxylic acid, triisobutyl ester of cyclohexane-1,2,4,5-tetracarboxylic acid, triglycol ester of cyclohexane-1,2,4,5-tetracarboxylic acid, tri-n-octyl ester of cyclohexane-1,2,4,5-tetracarboxylic acid, triisooctyl ester of cyclohexane-1,2,4,5-tetracarboxylic acid, tri-2-ethylhexyl ester of cyclohexane-1,2,4,5-tetracarboxylic acid, tri-n-nonyl ester of cyclohexane-1,2,4,5-tetracarboxylic acid, triisononyl ester of cyclohexane-1,2,4,5-tetracarboxylic acid, tri-n-decyl ester of cyclohexane-1,2,4,5-tetracarboxylic acid, triisododecyl ester of cyclohexane-1,2,4,5-tetracarboxylic acid, tri-n-undecyl ester of cyclohexane-1,2,4,5-tetracarboxylic acid, triisododecyl ester of cyclohexane-1,2,4,5-tetracarboxylic acid, tri-n-octadecyl ester of cyclohexane-1,2,4,5-tetracarboxylic acid, triisooctadecyl ester of cyclohexane-1,2,4,5-tetracarboxylic acid, tri-n-eicosyl ester of cyclohexane-1,2,4,5-tetracarboxylic acid, tricyclohexyl ester of cyclohexane-1,2,4,5-tetracarboxylic acid and tetramethyl ester of cyclohexane-1,2,4,5-tetracarboxylic acid, tetraethyl ester of cyclohexane-1,2,4,5-tetracarboxylic acid, tetra-n-propyl ester of cyclohexane-1,2,4,5-tetracarboxylic acid, tetra-n-butyl ester of cyclohexane-1,2,4,5-tetracarboxylic acid, tetra-tert-butyl ester of cyclohexane-1,2,4,5-tetracarboxylic acid, tetraisobutyl ester of cyclohexane-1,2,4,5-tetracarboxylic acid, tetraglycol ester of cyclohexane-1,2,4,5-tetracarboxylic acid, tetra-n-octyl ester of cyclohexane-1,2,4,5-tetracarboxylic acid, tetraisooctyl ester of cyclohexane-1,2,4,5-tetracarboxylic acid, tetra-2-ethylhexyl ester of cyclohexane-1,2,4,5-tetracarboxylic acid, tetra-n-nonyl ester of cyclohexane-1,2,4,5-tetracarboxylic acid, tetraisononyl ester of cyclohexane-1,2,4,5-tetracarboxylic acid, tetra-n-decyl ester of cyclohexane-1,2,4,5-tetracarboxylic acid, tetraisodecyl ester of cyclohexane-1,2,4,5-tetracarboxylic acid, tetraisododecyl ester of cyclohexane-1,2,4,5-tetracarboxylic acid, tetra-n-undecyl ester of cyclohexane-1,2,4,5-tetracarboxylic acid, tetra-n-octadecyl ester of cyclohexane-1,2,4,5-tetracarboxylic acid, tetraisooctadecyl ester of cyclohexane-1,2,4,5-tetracarboxylic acid, tetra-n-eicosyl ester of cyclohexane-1,2,4,5-tetracarboxylic acid, tetracyclohexyl ester of cyclohexane-1,2,4,5-tetracarboxylic acid, di-n-hexyl ester of cyclohexane-1,2,4,5-tetracarboxylic acid, diisohexyl ester of cyclohexane-1,2,4,5-tetracarboxylic acid, tri-n-hexyl ester of cyclohexane-1,2,4,5-tetracarboxylic acid, triisohexyl ester of cyclohexane-1,2,4,5-tetracarboxylic acid, di-n-heptyl ester of cyclohexane-1,2,4,5-tetracarboxylic acid, diisoheptyl ester of cyclohexane-1,2,4,5-tetracarboxylic acid, tri-n-heptyl ester of cyclohexane-1,2,4,5-tetracarboxylic acid, triisoheptyl ester of cyclohexane-1,2,4,5-tetracarboxylic acid, di-n-pentyl ester of cyclohexane-1,2,4,5-tetracarboxylic acid, diisopentyl ester of cyclohexane-1,2,4,5-tetracarboxylic acid, tri-n-pentyl ester of cyclohexane-1,2,4,5-tetracarboxylic acid, triisopentyl ester of cyclohexane-1,2,4,5-tetracarboxylic acid, di-n-tridecyl ester of cyclohexane-1,2,4,5-tetracarboxylic acid, diisotridecyl ester of cyclohexane-1,2,4,5-tetracarboxylic acid, tri-n-tridecyl ester of cyclohexane-1,2,4,5-tetracarboxylic acid, triisotridecyl ester of cyclohexane-1,2,4,5-tetracarboxylic acid, di-n-dodecyl ester of cyclohexane-1,2,4,5-tetracarboxylic acid, diisoundecyl ester of cyclohexane-1,2,4,5-tetracarboxylic acid, tri-n-dodecyl ester of cyclohexane-1,2,4,5-tetracarboxylic acid, triisoundecyl ester of cyclohexane-1,2,4,5-tetracarboxylic acid, triisopropyl ester of cyclohexane-1,2,4,5-tetracarboxylic acid, tetra-n-hexyl ester of cyclohexane-1,2,4,5-tetracarboxylic acid, tetraisohexyl ester of cyclohexane-1,2,4,5-tetracarboxylic acid, tetra-n-heptyl ester of cyclohexane-1,2,4,5-tetracarboxylic acid, tetraisoheptyl ester of cyclohexane-1,2,4,5-tetracarboxylic acid, tetra-n-pentyl ester of cyclohexane-1,2,4,5-tetracarboxylic acid, tetraisopentyl ester of cyclohexane-1,2,4,5-tetracarboxylic acid, tetra-n-tridecyl ester of cyclohexane-1,2,4,5-tetracarboxylic acid, tetraisotridecyl ester of cyclohexane-1,2,4,5-tetracarboxylic acid, tetra-n-dodecyl ester of cyclohexane-1,2,4,5-tetracarboxylic acid, tetraisoundecyl ester of cyclohexane-1,2,4,5-tetracarboxylic acid, tetraisopropyl ester of cyclohexane-1,2,4,5-tetracarboxylic acid;

anhydrides of cyclohexane-1,2-dicarboxylic acid, cyclohexane-1,2,4-tricarboxylic acid, cyclohexane-1,2,3-tricarboxylic acid and cyclohexane-1,2,4,5-tetracarboxylic acid.

For the purposes of the present invention, other suitable compounds are the esters of cyclohexane-1,2-dicarboxylic acid disclosed in WO 99/32427 and again listed below:

mixed esters of cyclohexane-1,2-dicarboxylic acid with C₁ to C₁₃ alcohols; (taken from the claims)

di(isopentyl)esters of cyclohexane-1,2-dicarboxylic acid obtainable via hydrogenation of di(isopentyl)phthalate with the Chemical Abstracts Registry Number (hereinafter: CAS No.) 84777-06-0;

di(isoheptyl)esters of cyclohexane-1,2-dicarboxylic acid obtainable via hydrogenation of di(isoheptyl)phthalate with the CAS No. 71888-89-6;

di(isononyl)esters of cyclohexane-1,2-dicarboxylic acid obtainable via hydrogenation of a di(isononyl)phthalate with the CAS No. 68515-48-0;

di(isononyl)esters of cyclohexane-1,2-dicarboxylic acid obtainable via hydrogenation of a di(isononyl)phthalate with the CAS No. 28553-12-0, based on n-butene;

di(isononyl)esters of cyclohexane-1,2-dicarboxylic acid obtainable via hydrogenation of a di(isononyl)phthalate with the CAS No. 28553-12-0, based on isobutene;

a 1,2-di-C₉ ester of cyclohexanedicarboxylic acid obtainable via hydrogenation of a di(nonyl)phthalate with the CAS No. 68515-46-8;

a di(isodecyl)ester of cyclohexane-1,2-dicarboxylic acid obtainable via hydrogenation of a di(isodecyl)phthalate with the CAS No. 68515-49-1;

a 1,2-di-C₇₋₁₁ ester of cyclohexane dicarboxylic acid obtainable via hydrogenation of the corresponding ester of phthalic acid with the CAS No. 68515-42-4;

a 1,2-di-C₇₋₁₁ ester of cyclohexane dicarboxylic acid obtainable via hydrogenation of di-C₇₋₁₁ phthalates with the following CAS Nos.

111 381-89-6,

111 381 90-9,

111 381 91-0,

68515-44-6,

68515-45-7, and

3648-20-2;

a 1,2-di-C₉₋₁₁ ester of cyclohexanedicarboxylic acid obtainable via hydrogenation of a di-C₉₋₁₁ phthalate with the CAS No. 98515-43-6;

a 1,2-di(isodecyl)ester of cyclohexanedicarboxylic acid obtainable via hydrogenation of a di(isodecyl)phthalate composed mainly of di(2-propylheptyl)phthalate;

a 1,2-di-C₇₋₉ ester of cyclohexanedicarboxylic acid obtainable via hydrogenation of the corresponding ester of phthalic acid of the branched-chain or linear C₇₋₉-alkyl ester groups; corresponding phthalates which can be used, for example, as starting materials have the following CAS Nos.:

di-C₇₋₉-alkyl phthalate with the CAS No. 111 381-89-6;

di-C₇-alkyl phthalate with the CAS No. 68515-44-6; and

di-C₉-alkyl phthalate with the CAS No. 68515-45-7.

The content of WO 99/32427 relating inter alia to these compounds listed immediately above and to the preparation of benzenepolycarboxylic acids using specific macroporous catalysts is incorporated in its entirety into the present application by way of reference.

According to the invention it is also possible to use hydrogenation products of mixed phthalates with C₁₀ alcohols and with C₁₃ alcohols, these being described in DE-A 10032580.7.

Other suitable compounds for the purposes of the present invention are the hydrogenation products of the commercially available benzenecarboxylic esters with the trade names Jayflex DINP (CAS No. 68515-48-0), Jayflex DIDP (CAS No. 68515-49-1), Palatinol 9-P, Vestinol 9 (CAS No. 28553-12-0), TOTM-I (CAS No. 3319-31-1), Linplast 68-TM, Palatinol N (CAS No. 28553-12-0), Jayflex DHP (CAS No. 68515-50-4), Jayflex DIOP (CAS No. 27554-26-3), Jayflex UDP (CAS No. 68515-47-9), Jayflex DIUP (CAS No. 85507-79-5), Jayflex DTDP (CAS No. 68515-47-9), Jayflex L9P (CAS No. 68515-45-7), Jayflex L911P (CAS No. 68515-43-5), Jayflex L11P (CAS No. 3648-20-2), Witamol 110 (CAS No. 90193-91-2), Witamol 118 (di-n-C₈-C₁₀-alkyl phthalate), Unimoll BB (CAS No. 85-68-7), Linplast 1012 BP (CAS No. 90193-92-3), Linplast 13 XP (CAS No. 27253-26-5), Linplast 610 P (CAS No. 68515-51-5), Linplast 68 FP (CAS No. 68648-93-1) and Linplast 812 HP (CAS No. 70693-30-0), Palatinol AH (CAS No. 117-81-7), Palatinol 711 (CAS No. 68515-42-4), Palatinol 911 (CAS No. 68515-43-5), Palatinol 11 (CAS No. 3648-20-2), Palatinol Z (CAS No. 26761-40-0) and Palatinol DIPP (CAS No. 84777-06-0).

Particularly preferred cosmetic compositions comprise diesters of 1,2-dicyclohexanecarboxylic acid. Preferred ester groups are straight-chain or branched alkyl groups having 8 to 13 carbon atoms. For example, it may be diisononylcyclohexane-1,2-dicarboxylate, which is also commercially available under the name Hexamoll® DINCH (BASF AG).

The cyclohexanepolycarboxylic acid derivatives are preferably prepared according to the process disclosed in WO 99/32427. This process comprises the hydrogenation of a benzenepolycarboxylic acid or of a derivative thereof, or the hydrogenation of a mixture of two or more thereof, by bringing the benzenepolycarboxylic acid or the derivative thereof or the mixture of two or more thereof into contact with a hydrogen-comprising gas in the presence of a catalyst which comprises, as active metal, at least one metal of the 8th transition group of the Periodic Table of the Elements alone or together with at least one metal of the 1st or 7th transition group of the Periodic Table of the Elements, applied to a support which has macropores.

In one preferred embodiment, the support has an average pore diameter of at least 50 nm and a BET surface area of not more than 30 m²/g, and the amount of the active metal is from 0.01 to 30% by weight, based on the total weight of the catalyst.

In another embodiment, use is made of a catalyst where the amount of the active metal is from 0.01 to 30% by weight, based on the total weight of the catalyst, and where from 10 to 50% of the pore volume of the support comprises macropores with a pore diameter in the range from 50 nm to 10 000 nm, and from 50 to 90% of the pore volume of the support comprises mesopores with a pore diameter in the range from 2 to 50 nm, the total of the proportions of the pore volumes being 100%.

In another embodiment, the catalyst comprises from 0.01 to 30% by weight, based on the total weight of the catalyst, of an active metal, applied to a support, where the support has an average pore diameter of at least 0.1 μm and a BET surface area of at most 15 m²/g. In principle, use may be made of any of the supports which have macropores, i.e. supports which have exclusively macropores, or else those which comprise not only macropores but also meso- and/or micropores.

The active metal used may in principle be any of the metals of the 8th transition group of the Periodic Table of the Elements. It is preferable for the active metals used to be platinum, rhodium, palladium, cobalt, nickel or ruthenium, or a mixture of two or more thereof, and ruthenium is in particular used as active metal. Among the metals of the 1st or 7th, or the 1st and the 7th, transition group of the Periodic Table which can also be used, preference is given to the use of copper and/or rhenium, but in principle any of these metals may be used.

For the purposes of the present invention, the definitions of the terms “macropores” and “mesopores” are those given in Pure Appl. Chem., 45 p. 79 (1976), i.e. pores whose diameter is above 50 nm (macropores) or from 2 to 50 nm, (mesopores).

The content of the active metal is generally from 0.01 to 30% by weight, preferably from 0.01 to 5% by weight, particularly preferably from 0.1 to 5% by weight, based in each case on the total weight of the catalyst used.

The term “benzenepolycarboxylic acid or a derivative thereof” used here comprises any of the benzenepolycarboxylic acids themselves, such as, for example, phthalic acid, isophthalic acid, terephthalic acid, trimellitic acid, trimesic acid, hemimellitic acid, and pyromellitic acid, and derivatives thereof, and mention may be made in particular here of mono-, di-, tri- and tetraesters, in particular alkyl esters, and anhydrides.

Preferred embodiments of the suitable catalysts and preferably used compounds and reaction conditions are disclosed in WO 99/32427.

The present application thus further provides cosmetic compositions comprising at least one cyclohexanepolycarboxylic acid derivative, where the cyclohexanepolycarboxylic acid derivative can be prepared by the following process:
a) esterification of a benzenepolycarboxylic acid of the formula II 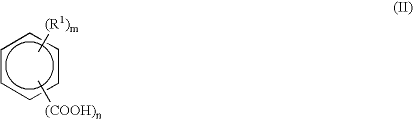
   where
R¹ is C₁-C₁₀-alkyl or C₃-C₈-cycloalkyl,
m is 0, 1, 2 or 3, and
n is 2, 3 or 4,
   with one or more alcohols of the formula
   R—OH
   where
R is C₁-C₃₀-alkyl, preferably C₁-C₂₀-alkyl, particularly preferably C₁-C₁₈-alkyl, very particularly preferably C₁-C₁₃-alkyl, especially C₈-C₁₃-alkyl,
   giving a benzenepolycarboxylic ester of the formula III 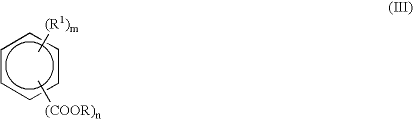
b) hydrogenation of the benzenepolycarboxylic ester of the formula III to give a corresponding cyclohexanepolycarboxylic ester.

Preferred embodiments of R¹, m, n, and R have been mentioned above in relation to the cyclohexanepolycarboxylic esters according to formula I.

One preferred embodiment of the hydrogenation of the benzenepolycarboxylic ester of the formula III (step b)) has been mentioned above.

Benzenepolycarboxylic acids whose use is preferred are phthalic acid, isophthalic acid, terephthalic acid, trimellitic acid, trimesic acid, hemimellitic acid, and pyromellitic acid. Phthalic acid is very particularly preferably used. The abovementioned acids are available commercially.

The alcohols used are preferably the alcohols corresponding to the radicals R of the cyclohexanepolycarboxylic acid derivatives of the formula I. Preference is therefore given to the use of linear or branched alcohols having C₁-C₁₃-alkyl radicals, particularly preferably having C₈-C₁₃-alkyl radicals. Each of the alcohols used for the esterification with the benzenepolycarboxylic acids may be the individual alcohol isomers corresponding to the abovementioned radicals R, or may be a mixture of different alcohols having isomeric alkyl radicals having the same number of carbon atoms, and/or may be a mixture of different alcohols having a different number of carbon atoms.

The alcohols or alcohol mixtures suitable for the reaction with the benzenepolycarboxylic acids may be prepared by any of the processes known to the person skilled in the art. Examples of suitable processes for the preparation of alcohols, or steps of processes used in the preparation of alcohols are:
hydroformylation with subsequent hydrogenation of the aldehydes formed, for example as disclosed in WO 92/13818, DE-A 2 009 505, DE-A 199 24 339, EP-A 1 113 034, WO 00/63151, WO 99/25668, JP-A 1 160 928, JP-A 03 083 935, JP-A 2000/053803, EP-A 0 278 407, EP-A 1 178 029, FR-A 1 304 144, JP-A 30 44 340, JP-A 30 44 341, JP-A 30 44 342, JP-A 0 40 36 251, GB-A 721,540, DE-A 195 304 14, JP-A 2001/049029, U.S. Pat. No. 2,781,396, U.S. Pat. No. 3,094,564, FR-A 1 324 873, JP-A 0 816 9854, U.S. Pat. No. 3,153,673, U.S. Pat. No. 3,127,451, U.S. Pat. No. 1,828,344, WO 2003/66642, WO 2003/18912, EP-A 0 424 767, WO 2002/68369, EP-A 0 366 089, JP-A 2001/002829, DE-A 100 35 617, DE-A 199 55 593, WO 2002/00580, EP-A 0 643 031, U.S. Pat. No. 2,876,264, JP-A 2000/319444, and DE-A 100 32 580;
hydrogenation of aldol products, for example as disclosed in DE-A 102 51 311, JP-A 05 194 761, U.S. Pat. No. 3,272,873, DE-A 3 151 086, JP-A 2001/322959, WO 98/03462, and EP-A 0 603 630;
hydration of alkenes, such as, for example, as disclosed in U.S. Pat. No. 5,136,108, EP-A 0 325 144, EP-A 0 325 143, DE-A 100 50 627, U.S. Pat. No. 4,982,022, GB-A 2,187,741, DE-A 36 28 008, U.S. Pat. No. 3,277,191, JP-A 2000/191 566, DE-A 854 377, DE-A 38 01 275, DE-A 39 25 217, JP-A 06 321 828, JP-A 02 088 536, JP-A 06 287 156, JP-A 06 287 155, JP-A 54 141 712, JP-A 08 283 186, JP-A 09 263 558, and U.S. Pat. No. 4,684,751;
hydrogenation of carboxylic acids and carboxylic esters, in particular fatty acids and fatty acid esters, for example as disclosed in U.S. Pat. No. 5,463,143, U.S. Pat. No. 5,475,159, WO 94/10112, CA 2,314,690, WO 94/06738, JP-A 06 065 125, and U.S. Pat. No. 3,361,832;
hydrogenation of unsaturated alcohols or of carbonyl compounds, for example as disclosed in EP-A 0 394 842, DE-A 1 269 605, WO 88/05767, FR-A 1,595,013, EP-A 0 326 674, BE-A 756 877, BE-A 757 561, DE-A 1 277 232, FR-A 1,499,041, and DE-A 1 276 620;
hydrogenation of epoxides, for example as disclosed in FR-A 1,508,939, GB-A 879 803, and DE-A 1 078 106;
processes comprising a telomerization step, for example as disclosed in EP-A 0 330 999, DE-A 1 138 751, U.S. Pat. No. 5,908,807, NE-6,603,884, and U.S. Pat. No. 3,091,628,
processes comprising an isomerization step, for example as disclosed in DE-A 42 28 887;
hydrolysis of sulfates, for examples as disclosed in GB-A 1,165,309;
reaction of dienes with amines, for example as disclosed in DE-A 44 31 528;
enzymatic preparation of alcohols, for example as disclosed in WO 93/24644;
selective hydrogenation of dienes, for example as disclosed in U.S. Pat. No. 3,203,998, DE-A 21 41 186, GB-A 2,093,025, JP-A 02 129 24, JP-A 1 122 8468, DE-A 195 44 133, WO 94/00410, GB-A 2,260,136, DE-A 44 10 746, and JP-A 08 176 036;
preparation of alcohols from nitrites, for example as disclosed in EP-A 0 271 092;
preparation of alcohols via reaction of alkynes, for example as disclosed in RU 205 9597-C1; and
hydrogenolysis of substituted tetrahydropyrans, for example as disclosed in GB 1,320,188.

The person skilled in the art is aware of other processes for preparing alcohols which can likewise be used for the preparation of alcohol mixtures or alcohols suitable for the esterification with benzenepolycarboxylic acids. As mentioned above, alcohols whose use is preferred are those which have C₁-C₁₃-alkyl radicals, particularly preferably C₈-C₁₃-alkyl radicals. In particular, the relatively long-chain C₈-C₁₃ alcohols and, respectively, alcohol mixtures comprising these alcohols are particularly preferably prepared via catalytic hydroformylation (also termed oxo reaction) of olefins and subsequent hydrogenation of the aldehydes formed. The person skilled in the art is aware of suitable hydroformylation processes, which are disclosed in the abovementioned documents. The alcohol mixtures and alcohols disclosed in the documents mentioned may be reacted with the abovementioned benzenepolycarboxylic acids to give the desired alkyl esters of benzenepolycarboxylic acid and, respectively, mixtures of these esters.

C₅ alcohols or mixtures comprising C₅ alcohols, particularly preferably n-pentanol, may be prepared, by way of example, by hydroformylation of butadiene in the presence of an aqueous solution of a rhodium compound and of a phosphine as catalyst. An example of this process is disclosed in EP-A 0 643 031.

Suitable C₇ alcohol mixtures which may be used for the esterification with the benzenepolycarboxylic acids are disclosed by way of example in JP-A 2000/319 444. The C₇ alcohol mixture is prepared via hydroformylation with subsequent hydrogenation of the aldehydes formed.

Mixtures comprising C₈ alcohols and processes for their preparation are disclosed by way of example in GB-A 721 540, which describes a process for the preparation of isooctyl alcohols starting from heptenes by means of hydroformylation and subsequent hydrogenation. Other documents which disclose the preparation of C₇ alcohols or of mixtures comprising these alcohols are DE-A 195 30 414, JP-A 2001/49029, U.S. Pat. No. 2,781,396, U.S. Pat. No. 3,094,564, FR-A 1,324,873, JP-A 08 169 854, U.S. Pat. No. 3,153,673, U.S. Pat. No. 3,127,451, and U.S. Pat. No. 1,828,344.

C₉ alcohols or mixtures comprising C₉ alcohols are preferably prepared via dimerization of butenes, hydroformylation of the resultant octenes, and subsequent hydrogenation of the resultant C₉ aldehyde.

Suitable processes and mixtures comprising C₉ alcohols are disclosed by way of example in WO 92/13818, DE-A 20 09 505, DE-A 199 24 339, EP-A 1 113 034, WO 2000/63151, WO 99/25668, JP-A 1 160 928, JP-A 03 083 935, JP-A 2000/053803, EP-A 0 278 407, and EP-A 1 178 029.

C₁₀ alcohols and mixtures comprising these alcohols are disclosed by way of example in WO 2003/66642, WO 2003/18912, EP-A 0 424 767, WO 2002/68369, EP-A 0 366 089, and JP-A 2001/002829.

C₁₂ alcohols or mixtures comprising C₁₂ alcohols, in particular trimethylnonanol, and a process for their preparation are disclosed by way of example in WO 98/03462.

C₁₃ alcohols, and also mixtures comprising these alcohols, are disclosed by way of example in DE-A 100 32 580, DE-A 199 55 593, and WO 2002/00580.

If the alkyl radicals R of the cyclohexanepolycarboxylic esters are C₁-C₄-alkyl radicals, these are obtained via reaction of the benzenepolycarboxylic acids of the formula II with methanol, ethanol, n-propanol, isopropanol, n-butanol, isobutanol, sec-butanol or tert-butanol. For the preparation of these benzenepolycarboxylic esters where R is 3 or 4, use may be made in each case of a mixture of the propanols or butanols mentioned, or of individual isomers. It is preferable to use individual isomers of the propanol or of the butanol. The preparation of the abovementioned C₁-C₄ alcohols is known to the person skilled in the art.

If the alkyl radicals R of the cyclohexanepolycarboxylic esters are C₅-C₁₃-alkyl radicals, preferably C₈-C₁₃-alkyl radicals, it is preferable to use C₅-C₁₃ alcohols, particularly preferably C₈-C₁₃-alcohols, which have degrees of branching (ISO index) which are generally from 0.1 to 4, preferably from 0.5 to 3, particularly preferably from 0.8 to 2, and in particular from 1 to 1.5, meaning that each of the alcohols is generally a mixture of different isomers. The ISO index is a dimensionless variable determined by means of gas chromatography.
Method: Capillary GC
Apparatus: Capillary gas chromatograph with autosampler, split/splitless injection system, and flame ionization detector (FID)
Chemicals:—MSTFA (N-methyl-N-trimethylsilyltrifluoroacetamide)
   Comparisons for determining retention times
Specimen preparation: 3 drops of the specimen are kept at 80° C. for 60 minutes in 1 ml of MSTFA
GC conditions: Capillary column: Ultra-1
   Length: 50 m
   Internal diameter: 0.25 mm
   Film thickness: 0.1 micrometer
   Carrier gas: helium
   Column inlet pressure: 200 psi, constant
   Split: 80 ml/min
   Septum flushing: 3 ml/min
   Oven temperature: 120° C., 25 min, isothermic
   Injector temperature: 250° C.
   Detector temperature: 250° C. (FID)
   Injection volume: 0.5 microliter

The C₅-C₁₃-alcohols, preferably C₈-C₁₃-alcohols, are prepared in accordance with the abovementioned processes. For the preparation of cyclohexanepolycarboxylic esters where R is 9, which are very particularly preferably used in the cosmetic compositions, it is particularly preferable to use a nonanol mixture in which 1 to 20% by weight, preferably 3 to 18% by weight, particularly preferably 5 to 16% by weight, of the nonanol mixture has no branching point, and from 10 to 90% by weight, preferably 15 to 80% by weight, particularly preferably 20 to 70% by weight has one branching point, and from 5 to 40% by weight, preferably 10 to 35% by weight, particularly preferably 15 to 30% by weight, has two branching points, and from 0.1 to 10% by weight, preferably 0.1 to 8% by weight, particularly preferably from 0.1 to 5% by weight, has three branching points, and from 0 to 4% by weight, preferably 0 to 3% by weight, particularly preferably 0.1 to 2% by weight, is other components. These other components are generally understood as meaning nonanols having more than three branching points, decanols, or octanols. Where the sum of the compounds mentioned gives 100% by weight.

One particularly preferred embodiment of a nonanol mixture used for the preparation of cyclohexanepolycarboxylic acid derivatives whose use is preferred has the following composition:
from 1.73 to 3.73% by weight, preferably from 1.93 to 3.53% by weight, particularly preferably from 2.23 to 3.23% by weight, of 3-ethyl-6-methylhexanol;
from 0.38 to 1.38% by weight, preferably from 0.48 to 1.28% by weight, particularly preferably from 0.58 to 1.18% by weight, of 2,6-dimethylheptanol;
from 2.78 to 4.78% by weight, preferably from 2.98 to 4.58% by weight, particularly preferably from 3.28 to 4.28% by weight, of 3,5-dimethylheptanol;
from 6.30 to 16.30% by weight, preferably from 7.30 to 15.30% by weight, particularly preferably from 8.30 to 14.30% by weight, of 3,6-dimethylheptanol;
from 5.74 to 11.74% by weight, preferably from 6.24 to 11.24% by weight, particularly preferably from 6.74 to 10.74% by weight, of 4,6-dimethylheptanol;
from 1.64 to 3.64% by weight, preferably from 1.84 to 3.44% by weight, particularly preferably from 2.14 to 3.14% by weight, of 3,4,5-trimethylhexanol;
from 1.47 to 5.47% by weight, preferably from 1.97 to 4.97% by weight, particularly preferably from 2.47 to 4.47% by weight, of 3,4,5-trimethylhexanol, 3-methyl-4-ethylhexanol, and 3-ethyl-4-methylhexanol;
from 4.00 to 10.00% by weight, preferably from 4.50 to 9.50% by weight, particularly preferably from 5.00 to 9.00% by weight, of 3,4-dimethylheptanol;
from 0.99 to 2.99% by weight, preferably from 1.19 to 2.79% by weight, particularly preferably from 1.49 to 2.49% by weight, of 4-ethyl-5-methylhexanol, and 3-ethylheptanol;
from 2.45 to 8.45% by weight, preferably from 2.95 to 7.95% by weight, particularly preferably from 3.45 to 7.45% by weight, of 4,5-dimethylheptanol, and 3-methyloctanol;
from 1.21 to 5.21% by weight, preferably from 1.71 to 4.71% by weight, particularly preferably from 2.21 to 4.21% by weight, of 4,5-dimethylheptanol;
from 1.55 to 5.55% by weight, preferably from 2.05 to 5.05% by weight, particularly preferably from 2.55 to 4.55% by weight, of 5,6-dimethylheptanol;
from 1.63 to 3.63% by weight, preferably from 1.83 to 3.43% by weight, particularly preferably from 2.13 to 3.13% by weight, of 4-methyloctanol;
from 0.98 to 2.98% by weight, preferably from 1.18 to 2.78% by weight, particularly preferably from 1.48 to 2.48% by weight, of 5-methyloctanol;
from 0.70 to 2.70% by weight, preferably from 0.90 to 2.50% by weight, particularly preferably from 1.20 to 2.20% by weight, of 3,6,6-trimethylhexanol;
from 1.96 to 3.96% by weight, preferably from 2.16 to 3.76% by weight, particularly preferably from 2.46 to 3.46% by weight, of 7-methyloctanol;
from 1.24 to 3.24% by weight, preferably from 1.44 to 3.04% by weight, particularly preferably from 1.74 to 2.74% by weight, of 6-methyloctanol;
from 25 to 35% by weight, preferably from 28 to 33% by weight, particularly preferably from 29 to 32% by weight, of other alcohols having 9 or 10 carbon atoms, the entirety of the components mentioned giving 100% by weight.

Another particularly preferred embodiment of a nonanol mixture used for the preparation of cyclohexanepolycarboxylic acid derivatives whose use is preferred has the following composition:
from 6.0 to 16.0% by weight, preferably from 7.0 to 15.0% by weight, particularly preferably from 8.0 to 14.0% by weight, of n-nonanol;
from 12.8 to 28.8% by weight, preferably from 14.8 to 26.8% by weight, particularly preferably from 15.8 to 25.8% by weight, of 6-methyloctanol;
from 12.5 to 28.8% by weight, preferably from 14.5 to 26.5% by weight, particularly preferably from 15.5 to 25.5% by weight, of 4-methyloctanol;
from 3.3 to 7.3% by weight, preferably from 3.8 to 6.8% by weight, particularly preferably from 4.3 to 6.3% by weight, of 2-methyloctanol;
from 5.7 to 11.7% by weight, preferably from 6.3 to 11.3% by weight, particularly preferably from 6.7 to 10.7% by weight, of 3-ethylheptanol;
from 1.9 to 3.9% by weight, preferably from 2.1 to 3.7% by weight, particularly preferably from 2.4 to 3.4% by weight, of 2-ethylheptanol;
from 1.7 to 3.7% by weight, preferably from 1.9 to 3.5% by weight, particularly preferably from 2.2 to 3.2% by weight, of 2-propylhexanol;
from 3.2 to 9.2% by weight, preferably from 3.7 to 8.7% by weight, particularly preferably from 4.2 to 8.2% by weight, of 3,5-dimethylheptanol;
from 6.0 to 16.0% by weight, preferably from 7.0 to 15.0% by weight, particularly preferably from 8.0 to 14.0% by weight, of 2,5-dimethylheptanol;
from 1.8 to 3.8% by weight, preferably from 2.0 to 3.6% by weight, particularly preferably from 2.3 to 3.3% by weight, of 2,3-dimethylheptanol;
from 0.6 to 2.6% by weight, preferably from 0.8 to 2.4% by weight, particularly preferably from 1.1 to 2.1% by weight, of 3-ethyl-4-methylhexanol;
from 2.0 to 4.0% by weight, preferably from 2.2 to 3.8% by weight, particularly preferably from 2.5 to 3.5% by weight, of 2-ethyl-4-methylhexanol;
from 0.5 to 6.5% by weight, preferably from 1.5 to 6% by weight, particularly preferably from 1.5 to 5.5% by weight, of other alcohols having 9 carbon atoms.

Where the sum of the components mentioned gives 100% by weight.

The present application also provides cyclohexanepolycarboxylic esters preparable by a process comprising the steps
a) esterification of a benzenepolycarboxylic acid of the formula II 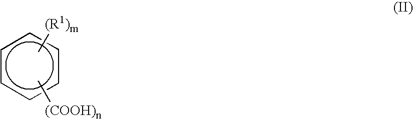
   where
R¹ is C₁-C₁₀-alkyl or C₃-C₈-cycloalkyl,
m is 0, 1, 2 or 3, and
n is 2, 3 or 4,
   with one or more alcohols of the formula
   R′—OH
   where
R′ is C₅-C₁₃-alkyl, preferably C₈-C₁₃-alkyls,
   where the alkyl radicals R′ have degrees of branching of from 0.8 to 3, preferably 0.9 to 2, particularly preferably 1 to 1.8 (ISO index),
   giving a benzenepolycarboxylic ester of the formula III′ 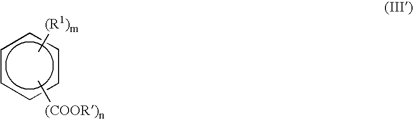
b) hydrogenation of the benzenepolycarboxylic ester of the formula III′ to give a corresponding cyclohexanepolycarboxylic ester.

Preferred alcohols R′—OH, in particular nonanol mixtures, are the abovementioned alcohols and alcohol mixtures. Preferred embodiments of R¹, m, and n have been mentioned above in relation to the cyclohexanepolycarboxylic esters according to formula I.

The cyclohexanepolycarboxylic esters according to the invention are particularly highly suitable as components in cosmetic compositions according to the present application.

The present application further provides cosmetic compositions comprising a cyclohexanepolycarboxylic acid derivative obtainable according to the process given above, i.e. the cyclohexanepolycarboxylic acid derivatives are obtainable via hydrogenation of a benzenepolycarboxylic acid or a derivative thereof, or via hydrogenation of a mixture of two or more thereof, by bringing the benzenepolycarboxylic acid or the derivative thereof or the mixture of two or more thereof into contact with a hydrogen-comprising gas in the presence of a catalyst which comprises, as active metal, at least one metal of the 8th transition group of the Periodic Table of the Elements alone or together with at least one metal of the 1st or 7th transition group of the Periodic Table of the Elements, applied to a support which has macropores.

Preferred embodiments of the cyclohexanepolycarboxylic acid derivative have already been given above.

Suitable cosmetic compositions are all customary cosmetic compositions. These are preferably fragrance compositions, fragrances for the treatment and care of the skin, such as soap, shower gel and bath gel, creams, lotions, sunscreen compositions, deodorants, compositions for the cleansing and care of the hair, such as hair shampoo, conditioner, hair gel, hair-setting compositions in the form of liquids or foams and further cleansing or care compositions for the hair. Furthermore, the cosmetic compositions comprise compositions for decorative application on the human body, such as cosmetic sticks, for example lipsticks, lipcare sticks, concealing sticks (concealers), blushers, eyeshadow pencils, lipliner pencils, eyeliner pencils, eyebrow pencils, corrector sticks, sunscreen sticks, antiacne sticks and comparable products, and also nail varnishes and further products for nail care.

In this connection, it is possible that the compositions mentioned for cleansing and caring for the skin and/or for cleansing and caring for the hair, and the other compositions mentioned to comprise the likewise listed fragrance compositions which in turn comprise at least one cyclohexanepolycarboxylic acid derivative used according to the invention. These fragrance compositions can, however, likewise be present in room deodorants, detergents or care compositions for textiles.

In a preferred embodiment, the compositions according to the invention are fragrance compositions. These fragrance compositions comprise at least one cyclohexanepolycarboxylic acid derivative according to the present application. Preferred and particularly preferred embodiments of the cyclohexanepolycarboxylic acid derivatives have already been specified above.

The components usually comprised in the cosmetic compositions are known to the person skilled in the art. The preferred fragrance compositions preferably comprise, besides the at least one cyclohexanepolycarboxylic acid derivative used according to the invention, one, some or all of the following compounds:
a) volatile phenolic compounds, such as isoamyl salicylate, benzyl salicylate and thyme oil red,
b) essential oils, such as geranium oil and patchouli oil,
c) citrus oils,
d) extracts and polymeric materials, such as benzoin siam resinoid, opoponax resinoid,
e) “synthetic” oils, such as bergamot 37 and 430, geranium 76 and pomeramsol 314,
f) aldehydes and ketones, such as β-methyl naphthyl ketone, p-t-butyl-α-methylhydroxycinnamaldehyde and p-t-amylcyclohexanone,
g) polycyclic compounds, such as coumarin and β-naphthyl methyl ether,
h) further esters (besides the cyclohexanepolycarboxylic acid derivatives used according to the invention, preferably cyclohexanepolycarboxylic esters) such as diethyl phthalate and phenylethyl phenylacetate, where preferably no aromatic polyalkyl esters such as diethyl phthalate are used,
i) alcohols, such as dimyrcetol, phenylethyl alcohol and tetrahydromuguol.

Furthermore, the fragrance compositions preferably comprise esters and essential oils of floral materials and fruits, citrus oils, aldehydes, resinoids, musk and other animal scents, such as natural isolates of the civet cat, of castoreum and of musk oxen.

The present application further provides a perfumed product chosen from the group consisting of perfume, Eau de Toilette, soap, shower gel, bath gel, shampoo, further haircare compositions, cosmetic preparations, body deodorant, room deodorant, detergents for textiles and care compositions for textiles comprising a fragrance composition according to the invention. Suitable perfumed products have already been specified above. Preference is given to using the fragrance composition according to the invention in body deodorants.

Further components of the perfumed products are known to the person skilled in the art.

A further preferred embodiment of the cosmetic compositions according to the invention relates to cosmetic compositions for decorative application on the human body. These compositions are preferably nail varnishes and/or further compositions for nail care, and also cosmetic sticks, such as lipsticks, lipcare sticks, concealing sticks (concealers), blushers, eyeshadow pencils, lipliner pencils, eyeliner pencils, eyebrow pencils, corrector sticks, sunscreen sticks, antiacne sticks and comparable products. The compositions according to the invention are preferably nail varnishes and lipsticks.

Suitable components of the specified cosmetic compositions for decorative application on the human body are known to the person skilled in the art.

In nail varnishes, the cyclohexanepolycarboxylic acid derivatives used according to the invention are preferably used as plasticizers and can thus replace the plasticizers which trigger allergies such as diethyl phthalate.

The present application further provides the use of at least one cyclohexanepolycarboxylic acid derivative in cosmetic compositions. Preferred cyclohexane polycarboxylic acid derivatives and cosmetic compositions have already been specified above. The use of such cyclohexanepolycarboxylic acid derivatives in cosmetic compositions is not known to date. The cyclohexanepolycarboxylic acid derivatives can satisfy a variety of tasks in the cosmetic compositions according to the invention. When used in fragrance compositions, the cyclohexanepolycarboxylic acid derivatives used according to the invention can serve as solvents; in nail varnishes the cyclohexanepolycarboxylic acid derivatives used according to the invention are suitable as plasticizers. It is important that the cyclohexanepolycarboxylic acid derivatives according to the invention are toxicologically safe and have an excellent dissolving power. Furthermore, the cyclohexanepolycarboxylic acid derivatives used according to the invention can have an emulsifying effect and also have an effect on slip and adhesion properties during processing to give, for example, cosmetic sticks.

Preferably, the cyclohexanepolycarboxylic acid derivatives used according to the invention are used in fragrance compositions, in particular in deodorants, and in cosmetic compositions for decorative application on the human body, in particular in cosmetic sticks and nail varnishes.

1. A cosmetic composition comprising at least one cyclohexanepolycarboxylic acid derivative of the formula (I) 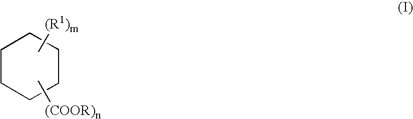
in which
R¹ is C₁-C₁₀-alkyl or C₃-C₈-cycloalkyl;
m is 0, 1, 2 or 3;
n is 2, 3 or 4, and
R is H or n-nonyl, isononyl, n-decyl, isodecyl, n-undecyl, isoundecyl, n-dodecyl, isododecyl, n-tridecyl, isotridecyl, stearyl, n-eicosyl, where at least one radical R is n-nonyl, isononyl, n-decyl, isodecyl, n-undecyl, isoundecyl, n-dodecyl, isododecyl, n-tridecyl, isotridecyl, stearyl, n-eicosyl.

2. The cosmetic composition according to claim 1, wherein the at least one cyclohexanepolycarboxylic acid derivative is chosen from the group consisting of ring-hydrogenated mono- and dialkyl esters of phthalic acid, isophthalic acid and terephthalic acid, ring-hydrogenated mono-, di- and trialkyl esters of trimellitic acid, of trimesic acid and of hemimillitic acid, or mono-, di, tri- and tetraalkyl esters of pyrromellitic acid, where the alkyl groups are chosen from n-nonyl, isononyl, n-decyl, isodecyl, n-undecyl, isoundecyl, n-dodecyl, isododecyl, n-tridecyl, isotridecyl, stearyl amd n-eicosyl and mixtures of two or more thereof.

3. The cosmetic composition according to claim 1, wherein the at least one cyclohexanepolycarboxylic acid derivative is chosen from the group consisting of
di(isononyl)esters of cyclohexane-1,2-dicarboxylic acid obtainable via hydrogenation of a di(isononyl)phthalate with the CAS No. 68515-48-0;
di(isononyl)esters of cyclohexane-1,2-dicarboxylic acid obtainable via hydrogenation of a di(isononyl)phthalate with the CAS No. 28553-12-0, based on n-butene;
di(isononyl)esters of cyclohexane-1,2-dicarboxylic acid obtainable via hydrogenation of a di(isononyl)phthalate with the CAS No. 28553-12-0, based on isobutene;
a 1,2-di-C₉ ester of cyclohexanedicarboxylic acid obtainable via hydrogenation of a di(nonyl)phthalate with the CAS No. 68515-46-8;
a di(isodecyl)ester of cyclohexane-1,2-dicarboxylic acid obtainable via hydrogenation of a di(isodecyl)phthalate with the CAS No. 68515-49-1;
a 1,2-di-C₉₋₁₁ ester of cyclohexanedicarboxylic acid obtainable via hydrogenation of a di-C₉₋₁₁ phthalate with the CAS No. 98515-43-6;
a 1,2-di(isodecyl)ester of cyclohexanedicarboxylic acid obtainable via hydrogenation of a di(isodecyl)phthalate composed mainly of di(2-propylheptyl)phthalate;
hydrogenation products of mixed phthalates with C₁₀ alcohols and with C₁₃ alcohols;
alkyl esters of cyclohexane-1,2-dicarboxylic acid, such as, for example, di-n-nonyl ester of cyclohexane-1,2-dicarboxylic acid, diisononyl ester of cyclohexane-1,2-dicarboxylic acid, di-n-decyl ester of cyclohexane-1,2-dicarboxylic acid, diisodecyl ester of cyclohexane-1,2-dicarboxylic acid, di-n-undecyl ester of cyclohexane-1,2-dicarboxylic acid, diisododecyl ester of cyclohexane-1,2-dicarboxylic acid, di-n-eicosyl ester of cyclohexane-1,2-dicarboxylic acid, diisoundecyl ester of cyclohexane-1,2-dicarboxylic acid, di-n-dodecyl ester of cyclohexane-1,2-dicarboxylic acid, di-n-tridecyl ester of cyclohexane-1,2-dicarboxylic acid, diisotridecyl ester of cyclohexane-1,2-dicarboxylic acid;
the hydrogenation products of the commercially available benzenecarboxylic esters with the trade names Jayflex DINP (CAS No. 68515-48-0), Jayflex DIDP (CAS No. 68515-49-1), Palatinol 9-P, Vestinol 9 (CAS No. 28553-12-0), Palatinol N (CAS No. 28553-12-0), Jayflex UDP (CAS No. 68515-47-9), Jayflex DIUP (CAS No. 85507-79-5), Jayflex DTDP (CAS No. 68515-47-9), Jayflex L9P (CAS No. 68515-45-7), Jayflex L911P (CAS No. 68515-43-5), Jayflex Li IP (CAS No. 3648-20-2), Linplast 1012 BP (CAS No. 90193-92-3), Linplast 13 XP (CAS No. 27253-26-5), Palatinol 911 (CAS No. 68515-43-5), Palatinol 11 (CAS No. 3648-20-2) and Palatinol Z (CAS No. 26761-40-0).

4. The cosmetic composition according to claim 1, wherein the at least one cyclohexanepolycarboxylic acid derivative is chosen from ring-hydrogenated phthalates which derive from an ester mixture comprising a mixed ester.

5. The cosmetic composition according to claim 1, comprising a cyclohexanepolycarboxylic acid derivative obtainable via hydrogenation of a benzenepolycarboxylic acid or of a derivative thereof, or via hydrogenation of a mixture of two or more thereof, by bringing the benzenepolycarboxylic acid or the derivative thereof or the mixture of two or more thereof into contact with a hydrogen-comprising gas in the presence of a catalyst which comprises, as active metal, at least one metal of the 8th transition group of the Periodic Table of the Elements alone or together with at least one metal of the 1st or 7th transition group of the Periodic Table of the Elements, applied to a support which has macropores.

6. The cosmetic composition according to claim 1, wherein the cosmetic composition is a fragrance composition.

7. The fragrance composition according to claim 6 comprising said at least one cyclohexanepolycarboxylic acid derivative, and one, some or all of the compounds specified below:
a) volatile phenolic compounds,
b) essential oils,
c) citrus oils,
d) extracts and polymeric materials,
e) “synthetic” oils
f) aldehydes and ketones,
g) polycyclic compounds,
h) further esters, and
i) alcohols.

8. A perfumed product chosen from the group consisting of perfume, Eau de Toilette, soap, shower gel, bath gel, shampoo, further haircare compositions, cosmetic preparations, body deodorant, room deodorant, detergents for textiles and care compositions for textiles comprising a fragrance composition according to claim 7.

9. The cosmetic composition according to claim 1, wherein the cosmetic composition is a composition for decorative application on the human body.

10. The cosmetic composition according to claim 9, wherein the cosmetic composition for decorative application on the human body is chosen from cosmetic sticks and nail varnishes.

11.-13. (canceled)

14. A cosmetic composition comprising at least one cyclohexanepolycarboxylic acid derivative, where the cyclohexanepolycarboxylic acid derivative can be prepared by the following process:
a) esterification of a benzenepolycarboxylic acid of the formula II 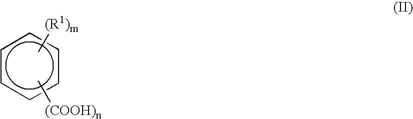
where
R¹ is C₁-C₁₀-alkyl or C₃-C₈-cycloalkyl,
m is 0, 1, 2 or 3, and
n is 2, 3 or 4,
with one or more alcohols of the formula
R—OH
where
R is n-nonyl, isononyl, n-decyl, isodecyl, n-undecyl, isoundecyl, n-dodecyl, isododecyl, n-tridecyl, isotridecyl, stearyl or n-eicosyl,
giving a benzenepolycarboxylic ester of the formula III 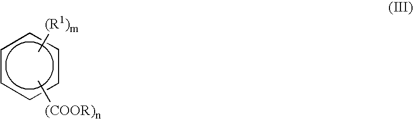
b) hydrogenation of the benzenepolycarboxylic ester of the formula III to give a corresponding cyclohexanepolycarboxylic ester.

15. A cyclohexanepolycarboxylic ester preparable by a process comprising the steps of
a) esterification of a benzenepolycarboxylic acid of the formula II 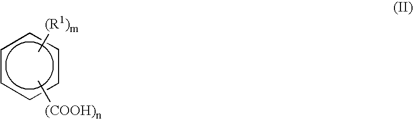
where
R¹ is C₁-C₁₀-alkyl or C₃-C₈-cycloalkyl,
m is 0, 1, 2 or 3, and
n is 2, 3 or 4,
with one or more alcohols of the formula
R′—OH
where
R′ is C₅-C₁₃-alkyl,
where the alkyl radicals R′ have degrees of branching of from 0.8 to 3 (ISO index),
giving a benzenepolycarboxylic ester of the formula III′ 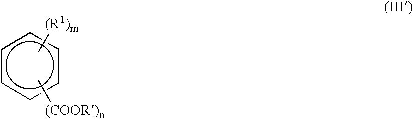
b) hydrogenation of the benzenepolycarboxylic ester of the formula III′ to give a corresponding cyclohexanepolycarboxylic ester.

16. A fragrance composition which comprises said at least one cyclohexanepolycarboxylic acid derivative according to claim 1.

17. A cosmetic composition for decorative application on the human body which comprises the at least one cyclohexanepolycarboxylic acid derivative according to claim 1.

18. The cosmetic composition as claimed in claim 17 wherein the composition is a cosmetic stick or nail varnish.

19. The cyclohexanepolycarboxylic ester as claimed in claim 15, wherein
R′ is C₈-C₁₃-alkyl,
wherein the alkyl radicals R′ have degrees of branching of from 0.9 to 2 (ISO index).

20. The cyclohexanepolycarboxylic ester as claimed in claim 19, wherein the alkyl radicals R′ have degrees of branching of from 1 to 1.8 (ISO index).
